(19) United States
(12) Patent Application Publication
(10) Pub. No.: US 2026/0048040 A1
(43) Pub. Date: Feb. 19, 2026

(54) MTA-COOPERATIVE PRMT5 INHIBITORS FOR USE IN THE TREATMENT OF CANCER

(71) Applicant: ASTRAZENECA AB, Sodertalje (SE)

(72) Inventors: James Thomas LYNCH, Cambridge (GB); Ho Man CHAN, Wilmington, DE (US)

(21) Appl. No.: 19/103,402

(22) PCT Filed: Aug. 14, 2023
(86) PCT No.: PCT/EP2023/072364

§ 371 Date: Feb. 12, 2025

Related U.S. Application Data
(60) Provisional application No. 63/397,996, filed on Aug. 15, 2022.

(51) Int. Cl. A61K 31/437; A61P 35/00; G01N 33/574

(52) U.S. Cl. CPC A61K 31/437; A61P 35/00; G01N 33/57484; C12Y 204/02028; G01N 2333/91142

(57) ABSTRACT

The present specification relates to methods of treatment of wild type MTAP gene cancers comprising administering a MTA synergistic PRMT5 inhibitor to a patient in need thereof.

The present specification claims benefit of priority to U.S. Provisional Application No. 63/397,996, filed 15 Aug. 2022, the content of which is hereby incorporated by reference in its entirety for all purposes.

This specification relates to methods for the treatment of cancer that comprise administration of a MTA synergistic PRMT5 inhibitor to a patient in need thereof, wherein the patient has a tumour that is wild type MTAP gene silenced i.e. a tumour that harbours wild type MTAP gene, but that nonetheless accumulates methylthioadenosine (MTA). One cancer type in which this profile has been found to be prevalent is Hodgkin Lymphoma (HL). The specification also relates to methods of identifying cancer patients who will benefit from treatment with an MTA-synergistic PRMT5 inhibitor.

Protein arginine methyltransferase 5 (PRMT5) is a member of the PRMT family of arginine methyltransferase enzymes that catalyse the addition of methyl groups to the guanidine motif of arginine residues, using S-adenosyl-L-methionine (SAM) as methyl donor. PRMT5 is a type II arginine methyltransferase that symmetrically dimethylates the guanidine group of arginine residues thus converting a guanidine NH₂ group of arginine to a NMe₂ group. PRMT5 methylates a number of diverse substrates including histone and non-histone proteins, and in so doing regulates processes such as RNA splicing, cellular proliferation and DNA repair. Significantly, PRMT5 is overexpressed in various cancer types and has been identified as a candidate for therapeutic intervention through the development of small molecules that inhibit PRMT5 methyltransferase activity (see e.g. Kim et al., (2020) Cell Stress 4 (8) 199-2151).

Cyclin dependent kinase inhibitor 2A (CDKN2A) is a tumour suppressor gene that is homozygously deleted in approximately 15% of cancers. Loss of the 9p21 chromosome locus (where CDKN2A resides) results in the co-deletion of additional genes including the gene MTAP encoding methylthioadenosine phosphorylase (MTAP). MTAP is a metabolic enzyme involved in methionine salvage. Loss of MTAP results in increased concentrations of the MTAP substrate methylthioadenosine (MTA) in CDKN2A/MTAP deleted cancer cells. MTA itself acts as a weak PRMT5 inhibitor and MTA accumulation in CDKN2A/MTAP deleted cancer cell lines accordingly leads to a partial inhibition of PRMT5 activity. Compromised PRMT5 activity renders CDKN2A/MTAP deleted cancer cells susceptible to further targeting of PRMT5, for example using short hairpin RNA (shRNA). A “collateral vulnerability” in cancer, where CDKN2A/MTAP deleted tumours may be selectively targeted through PRMT5 inhibition, has been identified (see Marjon et al., (2016) Cell Reports 15, 574-587; Mavrakis et al., (2016) Science 11; 351 (6278): 1208-13; Kryukov et al., (2016) Science 11; 351 (6278): 1214-8).

Recently, reports of MTA-synergistic PRMT5 inhibitors, i.e. PRMT5 inhibitors that bind to PRMT5 preferentially in the presence of MTA, have emerged (see e.g. WO2022/026892A1, WO2022/115377, WO2021/163344, WO2021/050915, WO2022/192745, WO2023/278564, WO2022/132914, WO2022/14619948, WO2023/036974, WO2023/081367, CN202310191381, CN116462676, CN116462677, WO2023/098439 and WO2021/086879). These MTA-synergistic PRMT5 inhibitors are designed to exploit the “collateral vulnerability” arising from CDKN2A/MTAP gene deletion described in the literature. Significantly, MTA-synergistic PRMT5 inhibitors exert a greater inhibitory effect on PRMT5 in environments where relatively high concentrations of MTA are present, such as that found in CDKN2A/MTAP deleted tumour cells, but not in healthy tissues where inhibition of PRMT5 would otherwise result in toxic side effects. Consequently, MTA-synergistic PRMT5 inhibitors should possess a high therapeutic index (and low off target toxicity) as their anti-proliferative activity will selectively manifest in the targeted, MTA rich, environment of CDKN2A/MTAP deleted tumour cells.

Hodgkin Lymphoma (HL) is a type of B cell lymphoma that accounts for about 15% of all lymphomas. Although the incidence of the HL is low in the general population, with 2-3 cases per 100,000 individuals with European ancestry (see e.g. J. M. Connors et al, Nature Rev Disease Primers, 6, Art.: 61 (2020)), it is one of the most common types of cancers in young adults. HL is seen also in elderly individuals, however with less frequency.

Histopathologically, 90-95% of HL cases are classified as classical HL (cHL) and the remaining 5-10% of the cases are classified as nodular lymphocyte-predominant HL (NLPHL). cHL has four subtypes namely nodular sclerosing (NSHL), mixed cellularity (MCHL), lymphocyte-rich (LRHL) and lymphocyte depleted (LDHL). LDHL is the most common subtype observed in all age groups.

HL is characterized by the presence of a few malignant cells surrounded by numerous immune effector cells in the tumour microenvironment. HL malignant cells are large mono or multinucleated cells with distinctive morphology and are derived from B cells. While the malignant cells in cHL are called Hodgkin and Reed-Sternberg (HRS) cells, the malignant cells in NLPHL are called lymphocyte predominant (LP) cells. HRS cells are characterized by CD30 expression. LP cells on the other hand are negative for CD30, but positive for CD20.

Malignant cells, either HRS or LP, make up only about 1% of the HL tumour cell composition. The rest of the HL tumours are mostly composed of non-cancerous immune cells and stromal cells. The low frequency of malignant cells in HL tumours makes it very challenging to analyse the genomic alterations in HRS and LP cells. Recently, methods such as laser capture microdissection and fluorescence associated cell sorting technologies enabled the efficient enrichment and genomic analysis of the HRS and LP cells by multiple research groups. In addition to these, methods based on capture of ctDNA have also been utilized successfully for the analysis of HL genomic landscape.

Radiation therapy and multiagent chemotherapy are utilized with a high chance of cure as for first line treatment of HL. For relapsed or refractory (R/R) disease, high dose chemotherapy and autologous hematopoietic stem cell transplantation are commonly used. Immunotherapeutic approaches using immune checkpoint inhibitors and antibody drug conjugates have given promising results for the treatment of R/R HL patients. Notwithstanding this, there is a need for alternative and improved approaches to the treatment of HL.

As described herein, it has been discovered that hypermethylation at, or around, the MTAP gene in certain tumour types that express wild type MTAP gene, and that are not MTAP gene deleted, can reduce or abrogate mRNA levels for MTAP, and as a result cause the tumour to accumulate MTA due to an absence of MTAP protein. Consequently, a hitherto unrealised opportunity for treating patients with certain MTAP wild type tumours has been revealed. Accordingly, it is an object of the present specification to provide new approaches to the treatment of tumours that harbour wild type MTAP gene, but that nonetheless accumulate MTA. In addition, the specification also provides a method for the identification of patients that are indicated for treatment with an MTA synergistic PRMT5 inhibitor, comprising the step of identifying from a sample obtained from the patient that the patient has a tumour that harbours wild type MTAP gene, but that nonetheless accumulates MTA in tumour cells.

According to a first aspect of the specification there is provided a method of treatment comprising administering a MTA synergistic PRMT5 inhibitor to a patient in need thereof, wherein the patient has been identified as having a tumour in which wild type MTAP gene is silenced. Tumours in which the wild type MTAP gene is silenced are those tumours that harbour intact, wild type, MTAP gene, but that nonetheless express reduced, or zero, MTAP mRNA or MTAP protein. As a result of their wild type MTAP gene silencing, such tumour cells have a reduced, or zero, capacity to phosphorylate MTA and thus accumulate MTA. As MTA synergistic PRMT5 inhibitors bind to, and inhibit, PRMT5 in concert with MTA and this in turn is potentiated in MTA rich environments a new “collateral vulnerability” opportunity is presented.

In a further aspect of the specification there is provided a method of treatment comprising administering a MTA synergistic PRMT5 inhibitor to a patient in need thereof, wherein the patient has been identified as having a tumour that harbours wild type MTAP gene and characteristically accumulates MTA.

In a further aspect of the specification there is provided a method of treatment comprising administering a MTA synergistic PRMT5 inhibitor to a patient in need thereof, wherein the patient has been identified as having a tumour that harbours wild type MTAP gene and characteristically accumulates MTA due MTAP gene silencing.

In a further aspect of the specification there is provided a method of treatment comprising administering a MTA synergistic PRMT5 inhibitor to a patient in need thereof, wherein the patient has been identified as having a tumour that harbours wild type MTAP gene and characteristically accumulates due MTAP gene silencing mediated by hypermethylation of MTAP.

In a further aspect the specification provides a method of treatment cancer comprising administering a MTA synergistic PRMT5 inhibitor to a patient in need thereof, wherein the patient has a tumour that harbours wild type MTAP gene but that accumulates MTA due to downregulation of MTAP at the protein level.

In a further aspect the specification provides a method of treatment cancer comprising administering a MTA synergistic PRMT5 inhibitor to a patient in need thereof, wherein the patient has a tumour that harbours wild type MTAP gene but that accumulates MTA due to downregulation of MTAP protein expression.

In a further aspect the specification provides a method of treating cancer comprising administering a MTA synergistic PRMT5 inhibitor to a patient in need thereof, wherein the patient's tumour harbours wild type MTAP gene but characteristically accumulates MTA due to epigenetic downregulation of MTAP mRNA.

In a further aspect the specification provides a method of treating cancer comprising administering a MTA synergistic PRMT5 inhibitor to a patient in need thereof, wherein the patient's tumour harbours wild type MTAP gene but accumulates MTA due to partial or complete silencing of MTAP protein expression.

In a further aspect the specification provides a method of treating cancer comprising administering a MTA synergistic PRMT5 inhibitor to a patient in need thereof, wherein the patient's tumour harbours wild type MTAP gene but accumulates MTA due to partial or complete silencing of MTAP protein expression due to epigenetic modification of the MTAP gene.

In a further aspect the specification provides a method of treating cancer comprising administering a MTA synergistic PRMT5 inhibitor to a patient in need thereof, wherein the patient's tumour harbours wild type MTAP gene but that accumulates MTA due to partial or complete silencing of MTAP protein expression due to hypermethylation of the MTAP gene.

In a further aspect the specification provides a method of treating cancer comprising administering a MTA synergistic PRMT5 inhibitor to a patient in need thereof, wherein the patient's tumour harbours wild type MTAP gene but that accumulates MTA due to downregulation of MTAP mRNA.

In a further aspect the specification provides a method of treating cancer comprising administering a MTA synergistic PRMT5 inhibitor to a patient in need thereof, wherein the patient's tumour harbours wild type MTAP gene but that accumulates MTA due to downregulation of MTAP mRNA caused by hypermethylation at, or around, the MTAP gene.

In a further aspect the specification provides a method of treating cancer comprising administering a MTA synergistic PRMT5 inhibitor to a patient in need thereof, wherein the patient's tumour harbours wild type MTAP gene but that accumulates MTA due to downregulation of MTAP mRNA caused by hypermethylation of the MTAP gene and/or a nearby gene such as CDKN2A or any other genomic location.

In a further aspect the specification provides a method of identifying a patient that will benefit from treatment with a MTA synergistic PRMT5 inhibitor, the method comprising analysing a sample obtained from the patient to confirm that the tumour harbours wild type MTAP gene but is nonetheless predisposed to accumulate MTA, optionally wherein the identification is made via performing an immunohistochemical assay that indicates relevant cell populations are MTAP protein deficient.

In aspects described above, the accumulation of MTA may be both in the nucleus and in the cytoplasm of relevant cells or may be localised in the nucleus of relevant cells.

In aspects described above, the accumulation of MTA may be determined by performing immunochemical staining for MTAP in a sample obtained from a patient.

In a further aspect the specification provides a method of identifying a patient that will benefit from treatment with a MTA synergistic PRMT5 inhibitor, the method comprising analysing a sample obtained from the patient and identifying that relevant tumour cells are wild type MTAP gene silenced.

In a further aspect the specification provides a method of identifying a patient that will benefit from treatment with a MTA synergistic PRMT5 inhibitor, the method comprising analysing a sample obtained from the patient and identifying that relevant tumour cells have reduced levels of MTAP protein or mRNA expression, optionally as identified by an immunohistochemical assay.

In a further aspect the specification provides a method of identifying a patient that will benefit from treatment with a MTA synergistic PRMT5 inhibitor, the method comprising analysing a sample obtained from the patient to confirm that the tumour i) harbours wild type MTAP gene and ii) that is MTAP deficient or MTAP null.

In a further aspect the specification provides a method of identifying a patient that will benefit from treatment with a MTA synergistic PRMT5 inhibitor, the method comprising analysing a sample obtained from the patient to confirm that the tumour i) harbours wild type MTAP gene and ii) that is MTAP mRNA null or deficient.

In a further aspect the specification provides a method of identifying a patient that will benefit from treatment with a MTA synergistic PRMT5 inhibitor, the method comprising performing an immunohistochemical assay for MTAP on a tumour sample obtained from the patient and identifying that relevant tumour cells are MTAP null or deficient.

In a further aspect the specification provides a method of treating cancer comprising the steps of i) identifying that the patient has a tumour that accumulates MTA as determined by performing a immunohistochemical assay for MTAP and ii) administering a MTA synergistic PRMT5 inhibitor to the patient.

In a further aspect the specification provides a MTA synergistic PRMT5 inhibitor for use in the treatment of cancer, wherein the cancer harbours wild type MTAP gene and accumulates MTA.

In a further aspect the specification provides a MTA synergistic PRMT5 inhibitor for use in the treatment of cancer, wherein the tumour harbours wild type MTAP gene and that is nonetheless MTAP null or deficient at the protein level.

In a further aspect the specification provides a kit comprising a MTA synergistic PRMT5 inhibitor and instructions for its use in the treatment of a cancer that harbours wild type MTAP gene and that is nonetheless MTAP null or deficient at the protein level.

So that the specification may be more readily understood reference to the following figures is made herein:

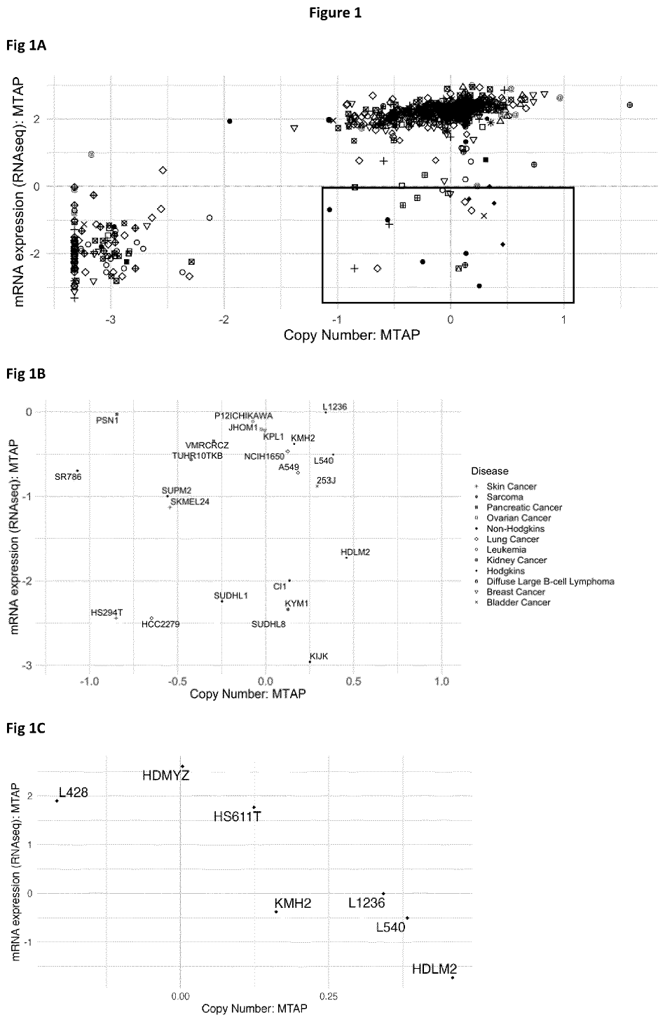

FIG. 1: A plot of MTAP mRNA expression vs MTAP copy number of the tumour cells in the Cancer Cell Line Encyclopedia (CCLE, https://sites.broadinstitute.org/ccle/). FIG. 1A presents the entire plot of the tumour cells in the CCLE. Samples within the boxed region have at least one copy of wild-type MTAP gene. FIG. 1B is the area of the plot of FIG. 1A that contains cells lines that harbour wild type MTAP gene, but that nonetheless have reduced MTAP gene expression as reflected by the low MTAP mRNA expression. FIG. 1C is a plot of the seven Hodgkin Lymphoma cell lines in the present in FIG. 1A.

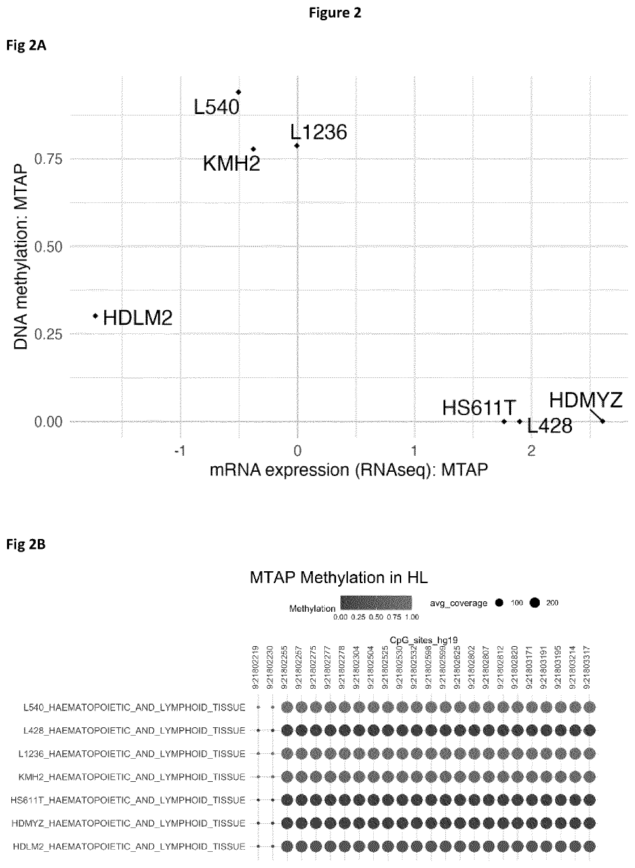

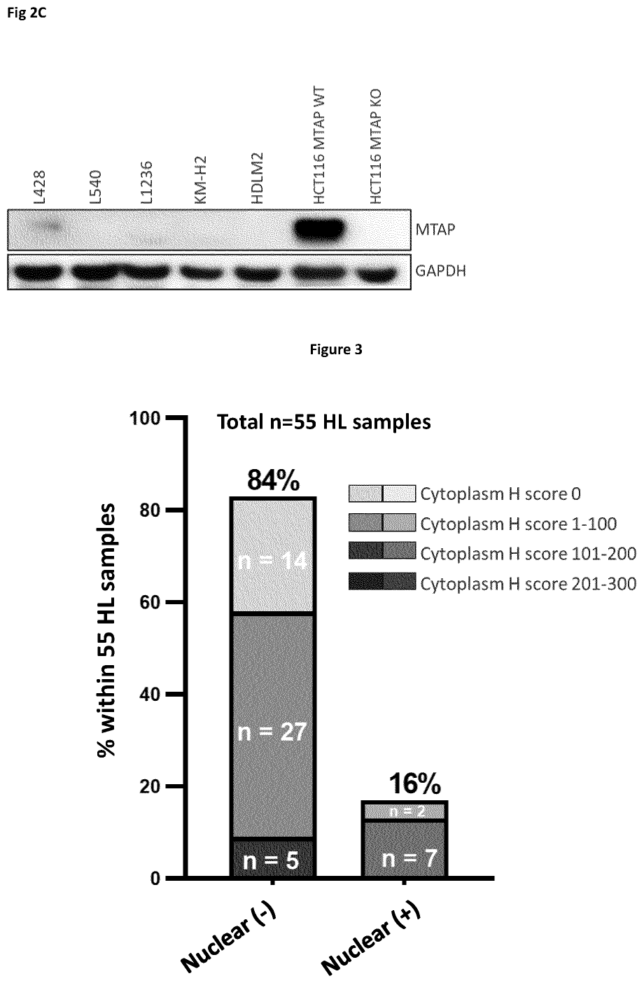

FIG. 2: FIG. 2A Plot of MTAP DNA methylation (y-axis) vs MTAP mRNA expression for the seven HL cell lines in the CCLE; FIG. 2B: methylation across the transcription site of the MTAP gene. X axis shows chromosome location of reduced representation bisulfite sequencing (RRBS) promotor methylation CpG clusters for MTAP promotor region (data acquired from Broad Institute https://data.broadinstitute.org/ccle/); FIG. 2C Western blot for MTAP, and GAPDH of four HL cells lines (L540, L1236, KMH2, HDLM2) that harbour wild type MTAP gene but that are gene silenced, alongside the non-MTAP silenced HL cell line L428. HCT116 colorectal cell line is included as a positive (MTAP wild type) and negative (MTAP KO) control.

FIG. 3: Plot illustrating the 84% of HL histopathology tumour samples staining for MTAP protein in the nucleus and cytoplasm. Of the 46 HL samples that are devoid of nuclear MTAP, 14 also lacked MTAP in the cytoplasm, 27 exhibit faint MTAP staining in the cytoplasm and 5 have more than +1 MTAP staining in the cytoplasm.

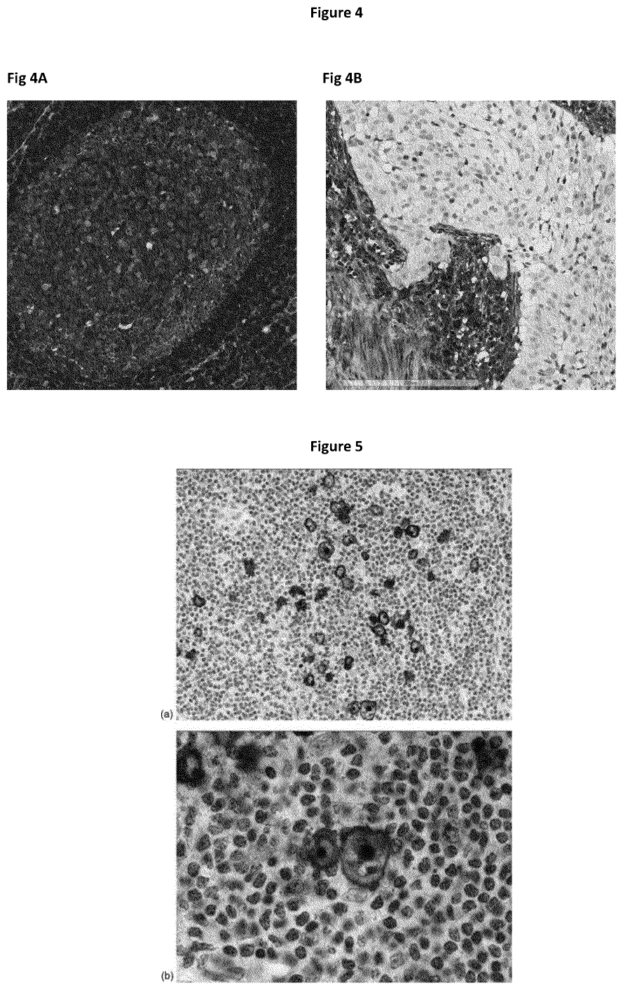

FIG. 4: Histopathology slide of normal tonsil tissue (FIG. 4A) and NSCLC (FIG. 4B). MTAP staining is seen across the cells in FIG. 4A. In contrast in FIG. 4B MTAP staining is confined to the tumour infiltrating lymphocytes (right hand side, dark region stain) while the NSCLC cells that extend from the top left-hand corner to the bottom right-hand corner of the image are free of staining for MTAP.

FIG. 5: Histopathology image taken from Kuppers, R. and Hansmann, M.-L., Int J Biochem & Cell Biol., 37 (3), 2005 p 511-17 showing the tumour clonal Hodgkin Reed/Sternberg (HRS) cells characteristic of Hodgkin Lymphoma stained with CD30 among the larger population of lymphoma cells.

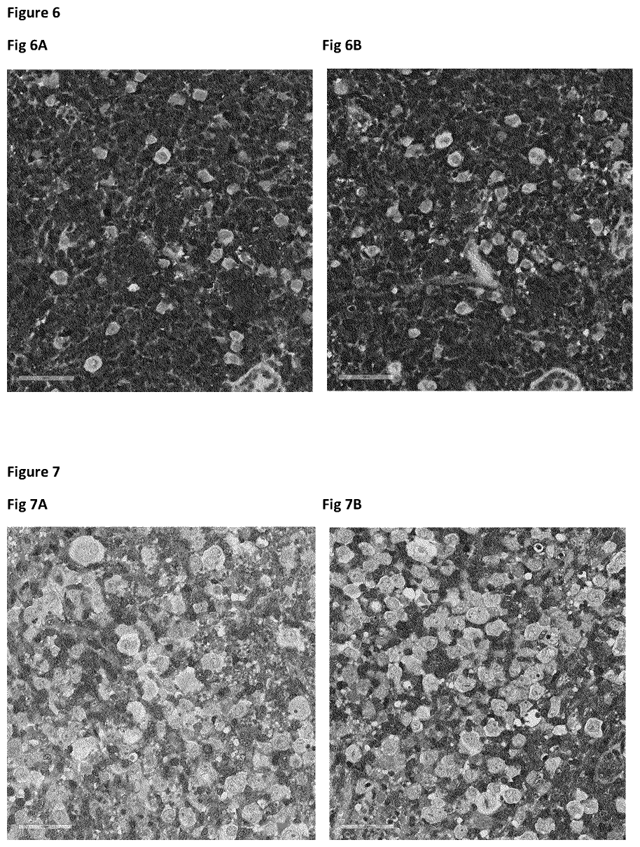

FIG. 6: Histopathology images obtained for Sample #: 243969-LN-1 (HL subtype: MC interfollicular) with MTAP antibody and casein containing diluent (FIG. 6A, 2 μg/ml mAb) or standard, casein free, diluent (FIG. 6B, 0.5 μg/mL). HRS cells are free from MTAP staining in both the nuclear and cytoplasmic compartments.

FIG. 7: Histopathology images obtained for Sample #: 243957-LN-1 (HL subtype: NS) with MTAP antibody and casein containing diluent (FIG. 7A, 2 μg/mL mAb) or standard, casein free, diluent (FIG. 7B, 0.5 μg/mL). A total absence of nuclear staining in HRS cells with some faint cytoplasmic staining for MTAP is observed.

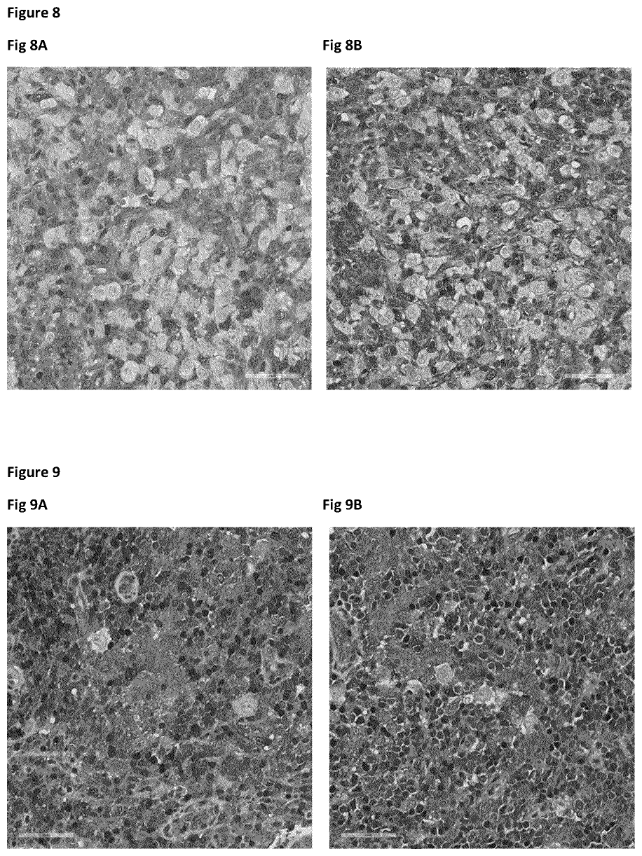

FIG. 8: Histopathology images obtained for Sample #: 243958-LN-1 (HL subtype: NS syncytial) with MTAP antibody and casein containing diluent (FIG. 8A, 2 μg/ml mAb) or standard, casein free, diluent (FIG. 8B, 0.5 μg/mL). A total absence of nuclear staining in HRS cells with some faint cytoplasmic staining for MTAP is observed.

FIG. 9: Histopathology images obtained for Sample #: 243965-LN-1 (HL subtype: MC) with MTAP antibody and casein containing diluent (FIG. 9A, 2 μg/mL mAb) or standard, casein free, diluent (FIG. 9B, 0.5 μg/mL). A total absence of nuclear staining in HRS cells with some faint cytoplasmic staining for MTAP is observed.

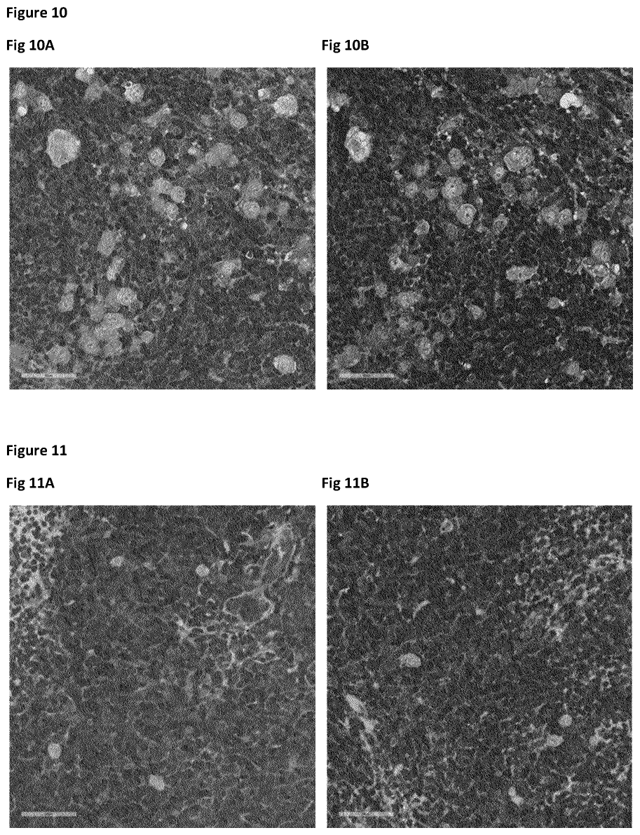

FIG. 10: Histopathology images obtained for Sample #: 243970-LN-1 (HL subtype: NS syncytial) in which the HRS cells are readily identifiable as the “light” areas, in this case the cells are assigned as exhibiting a total absence of nuclear staining in HRS cells with 1+ cytoplasmic staining. Staining performed with MTAP antibody and casein containing diluent (FIG. 10A, 2 μg/mL mAb) or standard, casein free, diluent (FIG. 10B, 0.5 μg/mL).

FIG. 11: Histopathology images obtained for Sample #: 243963-LN-1 (HL subtype: LRHL) in which the HRS cells are readily identifiable as the “light” areas, in this case the cells are assigned as exhibiting a total absence of nuclear staining in HRS cells with 1+ cytoplasmic staining. Staining performed with MTAP antibody and casein containing diluent (FIG. 11A, 2 μg/ml mAb) or standard, casein free, diluent (FIG. 11B, 0.5 μg/mL).

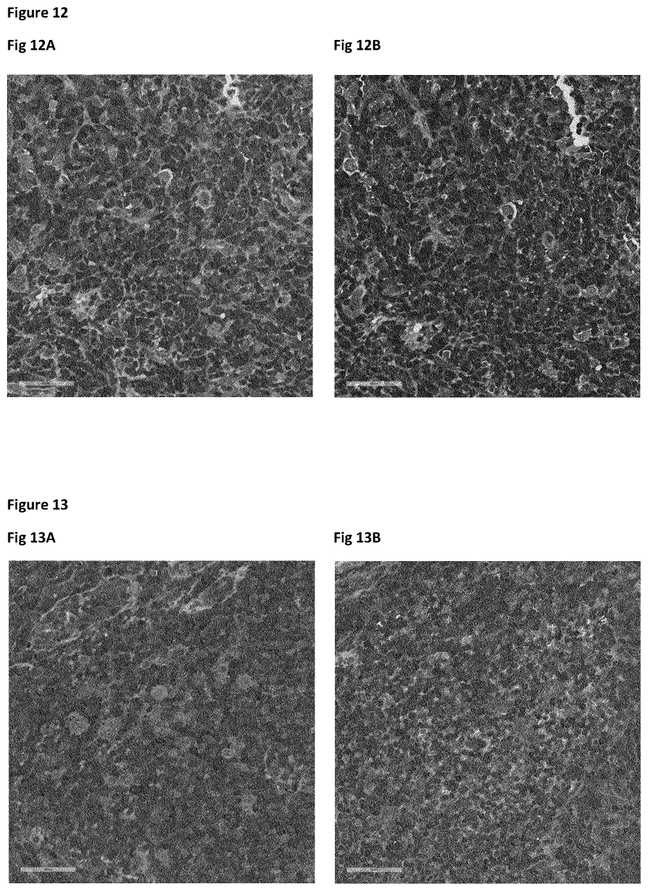

FIG. 12: Histopathology images obtained for Sample #: 243959-LN-1 (HL subtype: NS) in which the HRS cells are stain for MTAP in the nucleus and the cytoplasm of the HRS cells. Staining performed with MTAP antibody and casein containing diluent (FIG. 12A, 2 μg/ml mAb) or standard, casein free, diluent (FIG. 12B, 0.5 μg/mL).

FIG. 13: Histopathology images obtained for Sample #: 243961-LN-1 (HL subtype: NLPHL) in which the HRS cells are stained for MTAP in the nucleus and the cytoplasm of the LP (lymphocyte predominant) cells. Staining performed with MTAP antibody and casein containing diluent (FIG. 13A, 2 μg/ml mAb) or standard, casein free, diluent (FIG. 13B, 0.5 μg/mL).

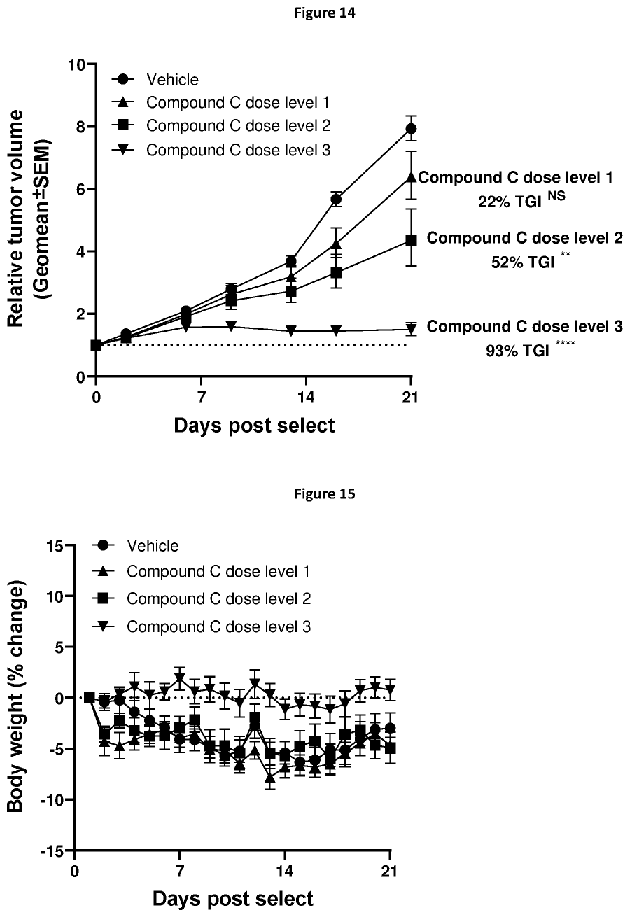

FIG. 14: Plot illustrating the effect of treatment with Compound C on the relative tumor volume in a L540 HL xenograft model.

FIG. 15: Plot illustrating the effect of treatment with Compound C on the body weight of mice in a L540 HL xenograft model.

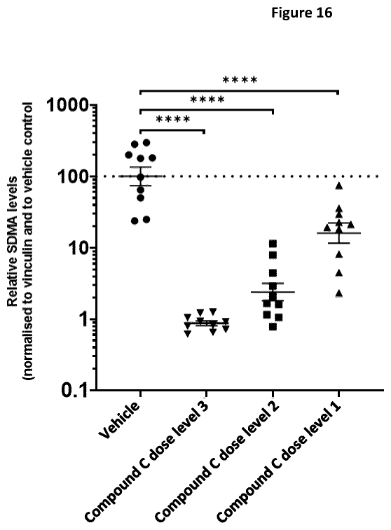

FIG. 16: Plot illustrating the effect of treatment with Compound C on SDMA protein levels in a L540 HL xenograft model.

The realisation of an opportunity for the application of MTA synergistic PRMT5 inhibitors in the treatment of tumours that are not CDKN2A/MTAP gene deleted stems from the novel observation that certain tumours harbour wild type MTAP gene but nonetheless accumulate MTA due to complete or partial silencing of MTAP gene. Based on the studies described herein, this particular phenotype appears to stem from hypermethylation at, or around, the MTAP gene in certain tumours that in turn causes a significant, or total, silencing of the MTAP gene or downregulation of MTAP gene expression that leads to reduced intratumoral concentration of MTAP protein. Functionally, hypermethylation at, or around, the MTAP gene reduces MTAP gene expression, with MTAP mRNA levels being reduced to low levels or being eliminated, i.e. the wild type MTAP gene is silenced. This in turn leads to a reduction or absence of MTAP protein in the tumour cell. MTAP is an enzyme that plays a major role in polyamine metabolism and that is important for the salvage of both adenine and methionine. In the context of treatment strategies for cancer, the absence of MTAP protein removes the clearance mechanism for methylthioadenosine (MTA) and causes accumulation of MTA. Tumour cells or tumours comprising relevant populations of cells that harbour wild type MTAP gene and yet still accumulate MTA are identified herein as tractable targets for treatment with MTA synergistic PRTM5 inhibitors (PRMT5 inhibitors that bind to PRMT5 in combination with MTA). In more detail, as MTA synergistic PRMT5 inhibitors only express their optimal activity in cells that have high concentrations of MTA, a selective cytotoxic effect can be exploited that avoids, or substantially reduces, the off-target toxicities associated with non-MTA selective PRMT5 inhibitors that has been observed in the clinic.

The realisation of an opportunity for selectively targeting certain tumours that harbour wild type MTAP gene with MTA synergistic PRMT5 inhibitors stems from an analysis of the Cancer Cell Line Encyclopaedia (CCLE, https://sites.broadinstitute.org/ccle/). In more detail, a search was performed for tumour cell lines in the CCLE that have low MTAP mRNA levels, a characteristic that had, before the work described herein, been associated with cells that have homozygous deletion of CDKN2A/MTAP. Accordingly, MTAP mRNA levels (as established by RNAseq) were plotted against MTAP (gene) copy number (see FIG. 1A). As expected, the search revealed a cluster of MTAP gene deleted cell lines to the bottom left-hand corner of FIG. 1A (NB the copy number scale on the x-axes of FIGS. 1A, 1B & 1C is the log ₂-1 of the copy number, thus cells with a log ₂-1 copy number of ≥−1 express at least one copy of wild type MTAP gene, while cells with a log ₂-1 copy number of ≤−2 are MTAP null, i.e. they do not express the MTAP gene) that have greatly reduced MTAP mRNA relative to cells harbouring wild type MTAP gene (those cells with a log ₂-1 MTAP copy number of −1) that cluster in the top right-hand corner of FIG. 1A. Unexpectedly, a group of cells in the CCLE were found to harbour wild type MTAP gene and are therefore found the right-hand side of the plot, but notwithstanding this exhibit MTAP mRNA levels equivalent to CDKN2A/MTAP gene deleted tumour cells (see cluster of cells at the bottom right-hand corner of FIG. 1A within the boxed area (MTAP copy number ≤−1, MTAP mRNA≤0). We refer to such cells that harbour wild type MTAP gene, but that nonetheless exhibit reduced MTAP mRNA expression as wild type MTAP gene silenced cells and, by extension tumours that comprise clonal tumour cells of this phenotype are referred to herein as wild type MTAP gene silenced tumours.

An expanded view of the population of tumour cells that are MTAP gene silenced i.e. those tumour cells that harbour wild type MTAP gene and that also exhibit low levels, or a total absence, of MTAP mRNA is presented in FIG. 1B. A table correlating the types of MTAP gene silenced tumour cells vs the total number of models as broken down on a tissue of origin or tumour type basis, and by prevalence of the MTAP gene silenced phenotype per tissue of origin is provided in Table 1. As can be seen from inspection of FIG. 1B and Table 1, 23 tumour cell lines from the CCLE were identified as both harbouring wild type MTAP gene and being MTAP silenced. Although in the panel many of the tumour cells were outliers in terms of prevalence in their tissue of origin or tumour type, a significant proportion of Hodgkin Lymphomas (4/7) and Non-Hodgkin Lymphomas (5/27) exhibited this MTAP silenced profile. The data for all of the seven Hodgkin Lymphoma cell lines in the CCLE are presented in FIG. 1C.

TABLE 1
Tumour cell models in the CCLE that harbour wild type MTAP
gene and that are MTAP gene silenced as evidenced by their
low MTAP mRNA levels, alongside the prevalence of this characteristic
on a tissue of origin or tumour type basis

| | | | % Prevalence |
|---|---|---|---|
| | No. of MTAP | Total No. | of MTAP |
| Tissue of origin | gene silenced | of models | gene silenced |
| or tumour type | models | in CCLE | models in CCLE |
| Bladder Cancer | 1 | 23 | 4.35 |
| Breast Cancer | 1 | 47 | 2.13 |
| Diffuse Large B-cell | 1 | 17 | 5.88 |
| Lymphoma (DLBCL) | | | |
| Hodgkin Lymphoma | 4 | 7 | 57.14 |
| Kidney Cancer | 2 | 22 | 9.09 |
| Leukemia | 1 | 76 | 1.32 |
| Lung Cancer | 3 | 160 | 1.88 |
| Non-Hodgkin | 5 | 27 | 18.52 |
| Lymphoma | | | |
| Ovarian Cancer | 1 | 43 | 2.33 |
| Pancreatic Cancer | 1 | 35 | 2.86 |
| Sarcoma | 1 | 26 | 3.85 |
| Skin Cancer | 2 | 49 | 4.08 |

This observation stimulated a further exploration on the origins of MTAP gene silencing with a view to establishing whether the “collateral vulnerability” opportunity presented by the availability of MTA synergistic PRMT5 inhibitors be exploited beyond non-CDKN2a/MTAP deleted tumours.

To understand the origins of MTAP gene silencing in tumour cell lines that harbour wild type MTAP gene we looked for unifying characteristics present across the 4/7 Hodgkin Lymphoma cell lines that harbour wild type MTAP gene but that are MTAP gene silenced relative to the 3 remaining, non-MTAP gene silenced, HL cell lines. It was established that 4 of the 7 HL cell lines, namely HDLM2, L540, KMH2 & L1236, that were gene silenced (see FIG. 2A) were also methylated across the transcription site of the MTAP gene (FIG. 2B) i.e. the MTAP gene was hypermethylated. The three HL cell lines that were unmethylated at MTAP gene in contrast expressed normal levels of MTAP mRNA and should therefore express MTAP at the protein level. A western blot for MTAP protein is shown in FIG. 2C and confirms that MTAP protein is only present in the MTAP mRNA expressing L428 cell line, while the HDLM2, L540, KMH2 & L1236 lines, in which the MTAP gene is hypermethylated, are MTAP protein null. The data therefore suggests that hypermethylation of MTAP gene causes MTAP gene silencing in numerous tumours.

Having established the likely, epigenetic, origins of MTAP gene silencing in HL cell lines, an experiment was performed to confirm that MTA synergistic PRMT5 inhibitors would be able to inhibit growth of the MTAP gene silenced HL cell lines and that should, accordingly, accumulate MTA. The activity of MTA synergistic PRMT5 inhibitors, Compound A, (S)-2-((5-Amino-6-fluoro-1H-pyrrolo[3,2-b]pyridin-2-yl)methyl)-5-fluoro-1′-(4-fluorobenzyl)spiro[isoindoline-1,3′-pyrrolidine]-2′,3-dione, and Compound C, (S)-2-((5-Amino-6-fluoro-1H-pyrrolo[3,2-b]pyridin-2-yl)methyl)-1′-(but-2-yn-1-yl)-5-fluorospiro[isoindoline-1,3′-pyrrolidine]-2′,3-dione were thus assessed for their ability to inhibit the growth of HDLM2, L540 and L1236 (all MTAP gene silenced) and L428 (intact MTAP expression), as well as in wild type and MTAP knock-out HCT116 cells. The activity of a non-selective PRMT5 inhibitor, Compound B (GSK3326595, as described in WO2015/198229A1, commercially available from e.g. medchemexpress.com-Cat No. HY-101563), across the same cells was assessed in parallel. The results obtained in these experiments are presented in Table 2.

TABLE 2
In vitro activity of MTA synergistic (Compounds A and C) and non-
selective (Compound B) PRMT5 inhibitors against the proliferation
of MTAP gene silenced and MTAP expressing Hodgkin Lymphoma cells
and wild type (wt) and MTAP knock out (KO) HCT116 cells.

| | MTAP mRNA level | | | | | |
|---|---|---|---|---|---|---|
| | low | | | high | low | high |
| | Cell Line | | | | | |
| | HDLM2 | L540 | L1236 | L428 | HCT116 | HCT116 |
| MTAP status | wt | wt | wt | wt | KO | wt |
| Compound A IC₅₀ (μM)* | 0.039 | 0.247 | 0.067 | 2.518 | 0.158 | >20 |
| Compound B IC₅₀ (μM)* | 0.093 | 0.157 | 0.014 | 0.583 | 0.074 | 0.075 |
| Compound C IC₅₀ (μM)* | 0.827 | 0.252 | 0.077 | 4.638 | 0.170 | 8.538 |

*average values from two or more experiments.

As can be seen from Table 2, all cell lines are sensitive to treatment with the non-selective PRMT5 inhibitor Compound B. In contrast, the MTA synergistic PRMT5 inhibitor Compound A expresses its activity predominantly in the MTAP gene silenced cell lines (HDLM2, L540 and L1236) and the MTAP knock out HCT116 cell line in which MTAP mRNA levels are low or absent and that will accordingly accumulate MTA due to the absence of, or reduced levels of, MTAP protein. Similarly, Compound C demonstrates preferential activity in the same cell lines as Compound A i.e. those which will accumulate MTA due to the absence of, or reduced levels of, MTAP protein. The potential to target tumours that harbour wild type MTAP gene, but that nonetheless accumulate MTA with MTA synergistic PRMT5 inhibitors is therefore demonstrated in vitro.

In order to investigate whether the prevalence of the MTAP gene wild type/MTAP silenced phenotype in the CCLE was representative of HL in the clinic we obtained a set of Hodgkin lymphoma samples and set out to assess whether, or not, a significant proportion of clinical Hodgkin Lymphoma samples would be deficient in MTAP protein expression and would therefore accumulate MTA. In order to perform this analysis an immunohistochemical (IHC) approach for detection of MTAP at the protein level was developed and applied to the analysis of the clinical samples. The results obtained from these experiments are summarised in FIG. 3 and Tables 3a and 3b.

TABLE 3a
Immunohistochemical analysis of MTAP protein in 15 Hodgkin Lymphoma clinical samples

| | | | | | MTAP Protein Expression | | | | |
|---|---|---|---|---|---|---|---|---|---|
| | | | | | | | | | Nuclear |
| Histology | | | | Clinical | | | | | H-Score |
| Number | Gender | Age* | Subtype | stage | Nuclear | Cytoplasmic | 0 | 1+ | (0-300) |
| 243969-LN-1 | M | 50 | Mixed | 4 | Negative | Negative | 100 | 0 | 0 |
| | | | cellularity | | | | | | |
| 243954-LN-1 | F | 22 | Nodular | 2 | Negative | Faint | 100 | 0 | 0 |
| | | | lymphocyte- | | | | | | |
| | | | rich classical | | | | | | |
| 243955-LN-1 | M | 35 | Nodular | | Negative | Faint | 100 | 0 | 0 |
| | | | lymphocyte- | | | | | | |
| | | | rich classical | | | | | | |
| 243957-LN-1 | F | 40 | Nodular | 3 | Negative | Faint | 100 | 0 | 0 |
| | | | sclerosing | | | | | | |
| 243958-LN-1 | F | 30 | Nodular | | Negative | Faint | 100 | 0 | 0 |
| | | | sclerosing | | | | | | |
| 243964-LN-1 | M | 61 | Nodular | 2 | Negative | Faint | 100 | 0 | 0 |
| | | | sclerosing | | | | | | |
| 243965-LN-1 | M | 59 | Mixed | 3a | Negative | Faint | 100 | 0 | 0 |
| | | | cellularity | | | | | | |
| 243967-LN-1 | M | 54 | Mixed | 1a | Negative | Faint | 100 | 0 | 0 |
| | | | cellularity | | | | | | |
| 243968-LN-1 | M | 43 | Nodular | 3 | Negative | Faint | 100 | 0 | 0 |
| | | | sclerosing | | | | | | |
| 243962-LN-1 | M | 78 | Nodular | | Negative | 1+ | 100 | 0 | 0 |
| | | | sclerosing | | | | | | |
| 243963-LN-1 | F | 56 | Nodular | 2 | Negative | 1+ | 100 | 0 | 0 |
| | | | lymphocyte- | | | | | | |
| | | | rich classical | | | | | | |
| 243966-LN-1 | F | 24 | Nodular | 2a | Negative | 1+ | 100 | 0 | 0 |
| | | | sclerosing | | | | | | |
| 243970-LN-1 | M | 48 | Nodular | 2 | Negative | 1+ | 100 | 0 | 0 |
| | | | sclerosing | | | | | | |
| 243959-LN-1 | F | 71 | Nodular | 1 | 1+ | 1+ | 0 | 100 | 100 |
| | | | sclerosing | | | | | | |
| 243961-LN-1 | F | 37 | Nodular | 1a | 1+ | 1+ | 0 | 100 | 100 |
| | | | lymphocyte | | | | | | |
| | | | predominance | | | | | | |

*= age at diagnosis. NB no 2+ or 3+ staining scores were obtained for nuclear or cytoplasmic MTAP in any of the 15 samples.

TABLE 3b
Immunohistochemical analysis of MTAP protein in 40 additional Hodgkin Lymphoma clinical samples

| | | MTAP protein expression | | | | | | |
|---|---|---|---|---|---|---|---|---|
| | | | | | | | | Nuclear |
| | | | | Nuclear stain intensity | | | | H score |
| Histology ID | Subtype | Nuclear | Cytoplasmic | 0 | 1+ | 2+ | 3+ | (0-300) |
| AVD-11YGH-2346A | Nodular Sclerosis | Negative | Negative | 100 | 0 | 0 | 0 | 0 |
| AVD-11YGH-2375A | Mixed Cellularity | Negative | Negative | 100 | 0 | 0 | 0 | 0 |
| AVD-11YGH-3749A | Mixed Cellularity | Negative | Negative | 100 | 0 | 0 | 0 | 0 |
| AVD-11YGH-3943A | Nodular Sclerosis | Negative | Negative | 100 | 0 | 0 | 0 | 0 |
| AVD-11YGH-4526A | Lymphocyte-rich | Negative | Negative | 100 | 0 | 0 | 0 | 0 |
| AVD-11YGH-5710A | Nodular Sclerosis | Negative | Negative | 100 | 0 | 0 | 0 | 0 |
| AVD-11YGH-6786A | Nodular Sclerosis | Negative | Negative | 100 | 0 | 0 | 0 | 0 |
| AVD-11YGH-7882A | Mixed Cellularity | Negative | Negative | 100 | 0 | 0 | 0 | 0 |
| AVD-11YGH-8050A | Nodular Sclerosis | Negative | Negative | 100 | 0 | 0 | 0 | 0 |
| AVD-11YGH-8310A | Nodular Sclerosis | Negative | Negative | 100 | 0 | 0 | 0 | 0 |
| AVD-11YGH-9116A | Mixed Cellularity | Negative | Negative | 100 | 0 | 0 | 0 | 0 |
| AVD-11YGH-9649A | Nodular Sclerosis | Negative | Negative | 100 | 0 | 0 | 0 | 0 |
| AVD-11YGH-9961A | Mixed Cellularity | Negative | Negative | 100 | 0 | 0 | 0 | 0 |
| AVD-11YGH-1003A | Nodular Sclerosis | Negative | 1+ | 100 | 0 | 0 | 0 | 0 |
| AVD-11YGH-1223A | Nodular Sclerosis | Negative | 1+ | 100 | 0 | 0 | 0 | 0 |
| AVD-11YGH-1747A | Nodular Sclerosis | Negative | 1+ | 100 | 0 | 0 | 0 | 0 |
| AVD-11YGH-2699A | Lymphocyte-rich | Negative | 1+ | 100 | 0 | 0 | 0 | 0 |
| AVD-11YGH-2933A | Nodular Sclerosis | Negative | 1+ | 100 | 0 | 0 | 0 | 0 |
| AVD-11YGH-3472A | Nodular Sclerosis | Negative | 1+ | 100 | 0 | 0 | 0 | 0 |
| AVD-11YGH-3807A | Mixed Cellularity | Negative | 1+ | 100 | 0 | 0 | 0 | 0 |
| AVD-11YGH-5497A | Nodular Sclerosis | Negative | 1+ | 100 | 0 | 0 | 0 | 0 |
| AVD-11YGH-5529A | Mixed Cellularity | Negative | 1+ | 100 | 0 | 0 | 0 | 0 |
| AVD-11YGH-6540A | Lymphocyte-depleted | Negative | 1+ | 100 | 0 | 0 | 0 | 0 |
| AVD-11YGH-7710A | Mixed Cellularity | Negative | 1+ | 100 | 0 | 0 | 0 | 0 |
| AVD-11YGH-7820A | Nodular Sclerosis | Negative | 1+ | 100 | 0 | 0 | 0 | 0 |
| AVD-11YGH-8772A | Nodular Sclerosis | Negative | 1+ | 100 | 0 | 0 | 0 | 0 |
| AVD-11YGH-9031A | Nodular Sclerosis | Negative | 1+ | 100 | 0 | 0 | 0 | 0 |
| AVD-11YGH-9487A | Nodular Sclerosis | Negative | 1+ | 100 | 0 | 0 | 0 | 0 |
| AVD-11YGH-7022A | Nodular Sclerosis | Negative | 1-2+ | 100 | 0 | 0 | 0 | 0 |
| AVD-11YGH-2529A | Mixed Cellularity | Negative | Faint | 100 | 0 | 0 | 0 | 0 |
| AVD-11YGH-2606A | Nodular Sclerosis | Negative | Faint | 100 | 0 | 0 | 0 | 0 |
| AVD-11YGH-4028A | Nodular Sclerosis | Negative | Faint | 100 | 0 | 0 | 0 | 0 |
| AVD-11YGH-7434A | Mixed Cellularity | Negative | Faint | 100 | 0 | 0 | 0 | 0 |
| AVD-11YGH-4681A | Mixed Cellularity | Faint | 1-2+ | 50 | 50 | 0 | 0 | 50 |
| AVD-11YGH-1627A | Lymphocyte-rich | 1+ | Faint | 0 | 100 | 0 | 0 | 100 |
| AVD-11YGH-7256A | Nodular Sclerosis | 1+ | 1+ | 0 | 100 | 0 | 0 | 100 |
| AVD-11YGH-5288A | Nodular Sclerosis | 1-2+ | 1-2+ | 0 | 50 | 50 | 0 | 150 |
| AVD-11YGH-1411A | Nodular Sclerosis | 2+ | 2+ | 0 | 0 | 100 | 0 | 200 |
| AVD-11YGH-3481A | Nodular Sclerosis | 2+ | 2+ | 0 | 0 | 100 | 0 | 200 |
| AVD-11YGH-5899A | Mixed Cellularity | 2+ | 2+ | 0 | 0 | 100 | 0 | 200 |

As can be seen from Tables 3a and 3b, IHC analysis revealed that 46 of 55 primary HL samples (84%) had nuclear MTAP loss, in other words, as judged by an expert histopathologist, there was a total absence of MTAP protein in the nuclei of the Hodgkin and Reed/Sternberg cells in these 46 Hodgkin Lymphoma samples. Interestingly, of the 46 samples that had MTAP loss, 32 expressed MTAP in the cytoplasm to a certain extent and were accorded either a +1 or a faint cytoplasmic MTAP score. Based on this sample set, it is apparent that a significant proportion of Hodgkin Lymphomas have reduced MTAP expression, a finding that correlates well with the data from the CCLE described above that is presented in FIG. 1.

To illustrate the IHC studies performed to arrive at the data above FIG. 4 shows the staining of a normal tonsil tissue sample with MTAP antibody that shows staining across the plate (FIG. 4A) whereas staining of a NSCLC sample (FIG. 4B) shows dark, stained areas (attributed as tumour infiltrating lymphocytes) and tumour cells that are substantially free of MTAP staining and that in the colour image show faint blue and light areas revealing the nuclei and cytoplasm.

To understand the significance of the histopathology images presented in FIG. 5 onward it is first important to recall that Hodgkin Lymphomas tissues comprise relatively small number of clonal tumour cells amongst a broader population of normal cells (see above). In more detail, Hodgkin and Reed/Sternberg cells, herein referred to as HRS cells, are the hallmark cells in Hodgkin Lymphoma and are large, often multinucleated cells with a characteristic and peculiar morphology and unusual immunophenotype, that do not resemble any normal cell in the body (see e.g. Küppers, R. and Hansmann, M.-L., Int J Biochem & Cell Biol., 37 (3), 2005 p 511-17). Hodgkin cells are characteristically mononucleated, while Reed/Sternberg cells are multinucleated. Despite their rarity in HL tissues, HRS cells are the clonal tumour cells of HL. HRS cells in nearly all cases of HL derive from B cells, and only rarely from T cells. Notably, the pattern of somatic mutations in their rearranged immunoglobulin V genes suggests that they are derived from pre-apoptotic germinal centre B cells. The pathogenesis of HL is still largely unresolved, but it is now clear that aberrant activation of several signalling pathways (such as the NFκB pathway) is of key importance for HRS cell survival. HRS or HRS-like cells are also found in several other diseases, e.g. as rare, intermingled, cells in some non-Hodgkin lymphomas and in infectious mononucleosis. To illustrate the prevalence of HRS cells in HL an image from the literature of an HL sample stained with CD30 is provided here as FIG. 5A with an enlarged image in FIG. 5B.

Turning to the data from the individual IHC analyses, FIG. 6 shows the histopathology image obtained for Sample #: 243969-LN-1 (HL subtype: MC interfollicular) with MTAP antibody and casein containing diluent (FIG. 6A, 2 μg/ml mAb) or standard, casein free, diluent (FIG. 6B, 0.5 μg/mL). All IHC images referred to in FIG. 6 onwards were obtained with the same conditions, i.e. with MTAP antibody and casein containing diluent (FIG. Xa where X=7 to 13) or standard, casein free, diluent (FIG. Xb) at the same concentrations. In this image the HRS cells are the light areas of the stained histopathology slide and the IHC analysis reveals a total absence of MTAP in the nuclear and cytoplasmic compartments of the HRS cells.

FIG. 7 presents data from Sample #: 243957-LN-1 (HL subtype: NS). Again, no nuclear staining for MTAP is observed in the HRS nuclei, but faint MTAP staining in the cytoplasmic compartment of the HRS cells is observed. FIG. 8 (Sample #: 243958-LN-1, HL subtype: NS syncytial) and FIG. 9 (Sample #: 243965-LN-1 HL subtype: MC) both likewise reveal a total absence of nuclear staining in HRS cells with some faint cytoplasmic staining.

FIG. 10 presents data from Sample #: 243970-LN-1 (HL subtype: NS syncytial) in which the HRS cells are readily identifiable as the “light” areas, in this case the cells are assigned as exhibiting a total absence of nuclear staining in HRS cells with 1+ cytoplasmic staining. The findings for the LRHL subtype Sample #: 243963-LN-1 (see FIG. 11) are likewise that the cells show a total absence of nuclear staining in HRS cells with 1+ cytoplasmic staining.

In contrast, IHC slides for Sample #: 243959-LN-1, HL subtype: NS and Sample #: 243961-LN-1, HL subtype: NLPHL are shown in FIGS. 12 and 13, respectively, with in each case MTAP staining being observed in the HRS cells.

Therefore, based on the 55 clinical samples analyzed, and in line with the observations drawn from the CCLE, we have identified that a significant proportion of Hodgkin Lymphomas accumulate MTA and that this MTA accumulation is due to MTAP gene silencing. The observation that loss of MTAP protein expression is very common in the nucleus (as seen in 46 of the 55 HL samples), but that residual levels of MTAP protein expression is present in 32 of the 46 sample that do not express MTAP in the nucleus clearly points to MTAP gene silencing, rather than MTAP gene deletion. Data in the Human Protein Atlas, confirms that MTAP protein expression is observed in both cytoplasmic and nuclear compartments in most tissues (see https://www.proteinatlas.org/ENSG00000099810-MTAP/tissue). Notwithstanding this, a “Protein Subcellular Localization Prediction Tool” (PSORT II) analysis, reveals no nuclear localization signal for the MTAP protein and predicts a proportional MTAP protein distributed in cytoplasm (78.3%), nucleus (17.4%) and endoplasmic reticulum (4.3%) (see https://psort.hgc.jp/form2.html). Interpolation from this prediction suggests that when MTAP protein levels are low, as dictated by partial MTAP gene silencing, detection of MTAP protein in the cytoplasm may still be possible when levels of MTAP protein in the nucleus are reduced below the limit of detection. In other words, a uniform % reduction in MTAP protein across the cell is more likely to result in MTAP protein levels below the limit of detection in the nucleus relative to the cytoplasm.

In the wider scientific literature reports suggest that if MTAP protein is expressed in a cell then depending on the expression levels, MTAP protein is expected to be observed in either both in cytoplasm and nucleus (when expression levels are high) or only in the cytoplasm (when expression levels are low), but never only in the nucleus.

In context of the data reported herein, we conclude that nuclear loss of MTAP protein in HL samples as detected by IHC is a strong indicator of overall MTAP protein deficiency, will lead to intracellular MTA accumulation and can be used as a surrogate marker for sensitivity to a MTA synergistic PRMT5 inhibitor.

The IHC experiments reported herein therefore confirm that a very significant proportion of HL clinical tumour samples are MTAP deficient. As these tumours will inevitably accumulate elevated concentrations of MTA, and given the availability of MTA synergistic PRMT5 inhibitors, a new method for treating HL, wherein the HL characteristically accumulates MTA, is revealed for the first time. This new “collateral vulnerability” opportunity should offer an efficacious option for the treatment of HL that characteristically has a favourable side effect profile since significant targeting of PRMT5 in healthy, MTAP protein expressing, cells should be minimal. Furthermore, this opportunity should be equally applicable to other tumour types, for example selected from those tumour types in which MTAP gene silencing is present disclosed in Table 1, that characteristically harbour wild type MTAP gene, but that are nonetheless of the MTAP gene silenced phenotype described for the first time herein.

In vivo experiments were performed to evaluate tumour growth inhibition and pharmacodynamic changes following treatment with the MTA synergistic PRMT5 inhibitor Compound C in the MTAP silenced L540 HL xenograft model (see Biological Example 1).

As noted above, in a first embodiment the present specification provides a method of treatment of cancer comprising administering a MTA synergistic PRMT5 inhibitor to a patient in need thereof, wherein the patient has been identified as having a tumour in which wild type MTAP gene has been silenced.

In embodiments, the MTAP gene silencing delivers a partial or total loss of MTAP gene expression protein in relevant tumour cells. Partial or total loss of MTAP gene expression can be established by immunohistochemical analysis or any other appropriate technique such as RT-qPCR that allows quantification of MTAP protein or mRNA in relevant cells or cell compartments.

In embodiments, the MTAP gene silencing delivers a partial or total loss of MTAP protein in the nucleus of relevant tumour cells.

In embodiments, the MTAP gene silencing delivers a partial or total loss of MTAP mRNA in relevant tumour cells. Partial or total loss of MTAP mRNA can be established by RNA-Seq, in situ hybridisation, or any other appropriate technique.

In embodiments, the MTAP gene silencing leads to a reduction of MTAP protein expression in the nucleus of tumour cells. In embodiments, the MTAP gene silencing leads to a reduction of MTAP protein expression in the nucleus of clonal tumour cells. In embodiments the tumour cells are Hodgkin Reed/Sternberg cells and the cancer is a Hodgkin Lymphoma.

In embodiments, the specification provides a method of treatment of cancer comprising administering a MTA synergistic PRMT5 inhibitor to a patient in need thereof, wherein the patient has been identified as having a tumour that harbours wild type MTAP gene and characteristically accumulates MTA.

In embodiments, the specification provides a method of treatment of cancer comprising administering a MTA synergistic PRMT5 inhibitor to a patient in need thereof, wherein the patient has been identified as having a tumour that harbours wild type MTAP gene and characteristically accumulates MTA due MTAP gene silencing.

In embodiments, the specification provides a method of treatment of cancer comprising administering a MTA synergistic PRMT5 inhibitor to a patient in need thereof, wherein the patient has been identified as having a tumour that harbours wild type MTAP gene and characteristically accumulates MTA in relevant tumour cells due MTAP gene silencing mediated by hypermethylation of MTAP.

In embodiments, the specification provides a method of treatment of cancer comprising administering a MTA synergistic PRMT5 inhibitor to a patient in need thereof, wherein the patient has been identified as having a tumour that harbours wild type MTAP gene and characteristically accumulates MTA in relevant tumour cells due to epigenetic modification of the MTAP gene.

In embodiments, the specification provides a method of treatment of cancer comprising administering a MTA synergistic PRMT5 inhibitor to a patient in need thereof, wherein the patient has a tumour that harbours wild type MTAP gene but that accumulates MTA in relevant tumour cells due to downregulation of MTAP at the protein level.

In embodiments, the specification provides a method of treatment of cancer comprising administering a MTA synergistic PRMT5 inhibitor to a patient in need thereof, wherein the patient has a tumour that harbours wild type MTAP gene but that accumulates MTA in relevant tumour cells due to downregulation of MTAP protein expression.

In embodiments, the specification provides a method of treatment of cancer comprising administering a MTA synergistic PRMT5 inhibitor to a patient in need thereof, wherein the patient's tumour harbours wild type MTAP gene but characteristically accumulates MTA in relevant tumour cells due to epigenetic downregulation of MTAP mRNA.

In embodiments, the specification provides a method of treatment of cancer comprising administering a MTA synergistic PRMT5 inhibitor to a patient in need thereof, wherein the patient's tumour harbours wild type MTAP gene but that accumulates MTA in relevant tumour cells due to partial or complete silencing of MTAP protein expression.

In embodiments, the specification provides a method of treatment of cancer comprising administering a MTA synergistic PRMT5 inhibitor to a patient in need thereof, wherein the patient's tumour harbours wild type MTAP gene but that accumulates MTA in relevant tumour cells due to partial or complete silencing of MTAP protein expression due to epigenetic modification of the MTAP gene.

In embodiments, the specification provides a method of treatment of cancer comprising administering a MTA synergistic PRMT5 inhibitor to a patient in need thereof, wherein the patient's tumour harbours wild type MTAP gene but that accumulates MTA in relevant tumour cells due to partial or complete silencing of MTAP protein expression due to hypermethylation of the MTAP gene.

In embodiments, the specification provides a method of treatment of cancer comprising administering a MTA synergistic PRMT5 inhibitor to a patient in need thereof, wherein the patient's tumour harbours wild type MTAP gene but that accumulates MTA in relevant tumour cells due to downregulation of MTAP mRNA.

In embodiments, the specification provides a method of treatment of cancer comprising administering a MTA synergistic PRMT5 inhibitor to a patient in need thereof, wherein the patient's tumour harbours wild type MTAP gene but that accumulates MTA in relevant tumour cells due to downregulation of MTAP mRNA caused by hypermethylation at, or around, the MTAP gene.

In embodiments, the specification provides a method of treatment of cancer comprising administering a MTA synergistic PRMT5 inhibitor to a patient in need thereof, wherein the patient's tumour harbours wild type MTAP gene but that accumulates MTA in relevant tumour cells due to downregulation of MTAP mRNA caused by hypermethylation of the MTAP gene and/or an adjacent gene such as CDKN2A.

In embodiments, the specification provides a method of treatment of cancer comprising administering a MTA synergistic PRMT5 inhibitor to a patient in need thereof, the method comprising analysing a sample obtained from the patient to confirm that the tumour harbours wild type MTAP gene but is nonetheless predisposed to accumulate MTA on the basis of a immunohistochemical assay that indicates relevant tumour cell populations are MTAP protein deficient.

In embodiments, the specification provides a method of treatment of cancer comprising administering a MTA synergistic PRMT5 inhibitor to a patient in need thereof, the accumulation of MTA may be both in the nucleus and in the cytoplasm of relevant tumour cells or may be localised in the nucleus of relevant cells.

In embodiments, the accumulation of MTA may be both in the nucleus and in the cytoplasm of relevant cells or may be localised or substantially localised to the nucleus of relevant cells.

In embodiments, the identification of the propensity of a tumour to accumulate MTA may be determined by performing immunochemical staining for MTAP in a sample obtained from a patient.

In embodiments, there is provided the use of an MTA synergistic PRMT5 inhibitor for the treatment of a cancer that is wild type MTAP gene silenced.

In embodiments, there is provided the use of an MTA synergistic PRMT5 inhibitor for the treatment of a cancer that is wild type MTAP gene silenced wherein the MTAP gene silencing
(a) delivers a partial or total loss of MTAP protein in relevant tumour cells; or
(b) delivers a partial or total loss of MTAP protein in the nucleus of relevant tumour cells; or
(c) leads to a reduction of MTAP protein expression in the nucleus of tumour cells; or
(d) leads to a reduction of MTAP protein expression in the nucleus of clonal tumour cells; or
(e) characteristically leads to accumulation of MTA in relevant tumour cells; or
(f) characteristically leads to accumulation of MTA in relevant tumour cells due MTAP gene silencing mediated by hypermethylation of MTAP; or
(g) characteristically leads to accumulation of MTA in relevant tumour cells due to epigenetic modification of the MTAP gene; or
(h) characteristically leads to accumulation of MTA in relevant tumour cells due to downregulation of MTAP at the protein level; or
(i) characteristically leads to accumulation of MTA in relevant tumour cells due to downregulation of MTAP protein expression; or
(j) results in epigenetically driven downregulation of MTAP mRNA; or
(k) leads to accumulation of MTA in relevant tumour cells due to partial or complete silencing of MTAP protein expression; or
(l) leads to accumulation of MTA in relevant tumour cells due to partial or complete silencing of MTAP protein expression due to epigenetic modification of the MTAP gene; or
(m) delivers a tumour that characteristically accumulates MTA due to partial or complete silencing of MTAP protein expression due to hypermethylation of the MTAP gene; or
(n) delivers a tumour that characteristically accumulates MTA due to downregulation of MTAP mRNA caused by hypermethylation at, or around, the MTAP gene; or
(o) delivers a tumour that characteristically accumulates MTA due to downregulation of MTAP mRNA caused by hypermethylation of the MTAP gene and/or an adjacent gene such as CDKN2A.

In embodiments, there is provided the use of an MTA synergistic PRMT5 inhibitor for the manufacture of a medicament for treatment of a cancer that is characterised by silencing of the wild type MTAP gene.

In embodiments, there is provided the use of an MTA synergistic PRMT5 inhibitor for the manufacture of a cancer that is wild type MTAP gene silenced wherein the MTAP gene silencing
(a) delivers a partial or total loss of MTAP protein in relevant tumour cells; or
(b) delivers a partial or total loss of MTAP protein in the nucleus in relevant tumour cells; or
(c) leads to a reduction of MTAP protein expression in the nucleus of tumour cells; or
(d) leads to a reduction of MTAP protein expression in the nucleus of clonal tumour cells; or
(e) characteristically leads to accumulation of MTA in relevant tumour cells; or
(f) characteristically leads to accumulation of MTA in relevant tumour cells due MTAP gene silencing mediated by hypermethylation of MTAP; or
(g) characteristically leads to accumulation of MTA in relevant tumour cells due to epigenetic modification of the MTAP gene; or
(h) characteristically leads to accumulation of MTA in relevant tumour cells due to downregulation of MTAP at the protein level; or
(i) characteristically leads to accumulation of MTA in relevant tumour cells due to downregulation of MTAP protein expression; or
(j) results in epigenetically driven downregulation of MTAP mRNA; or
(k) leads to accumulation of MTA in relevant tumour cells due to partial or complete silencing of MTAP protein expression; or
(l) leads to accumulation of MTA in relevant tumour cells due to partial or complete silencing of MTAP protein expression due to epigenetic modification of the MTAP gene; or
(m) delivers a tumour that characteristically accumulates MTA due to partial or complete silencing of MTAP protein expression due to hypermethylation of the MTAP gene; or
(n) delivers a tumour that characteristically accumulates MTA due to downregulation of MTAP mRNA caused by hypermethylation at, or around, the MTAP gene; or
(o) delivers a tumour that characteristically accumulates MTA due to downregulation of MTAP mRNA caused by hypermethylation of the MTAP gene and/or an adjacent gene such as CDKN2A.

As used herein and above, the term “relevant tumour cells” is used to refer to those cells that drive tumour growth and sustain tumour survival. An example of relevant tumour cells in each embodiment referring to “relevant tumour cells” are Hodgkin Reed/Sternberg cells as found in Hodgkin lymphoma.

In embodiments there is provided a method of identifying a patient that will benefit from treatment with an MTA synergistic PRMT5 inhibitor comprising the step of identifying that relevant tumour cells in a sample obtained from the patient exhibit reduced MTAP protein in their nuclei and, optionally also in their cytoplasm, by performing a immunohistochemical assay for MTAP protein on the sample.

In embodiments there is provided a method of identifying a patent that has a Hodgkin Lymphoma that will benefit from treatment with a MTA-synergistic PRMT5 inhibitor comprising the step of analysing MTAP expression in a tumour sample obtained from the patient and identifying that relevant tumour cells are wild MTAP gene silenced. In such embodiments the identification of the wild type MTAP gene silenced status made be made by a immunohistochemistry assay that shows MTAP protein expression in the nuclei and/or the cytoplasm of Hodgkin Reed/Sternberg cells is reduced or absent.

In embodiments relating to method of treatments set forth above the methods of treatment may also include the step of analysing a sample obtained from a patient suffering from cancer that they have a cancer that is wild type MTAP gene silenced. In such embodiments the determination of the wild type MTAP gene silenced may be made on the basis of a immunochemical assay for MTAP protein that reveals that MTAP protein expression in the nuclei and/or the cytoplasm of relevant tumour cells, for example in the case of Hodgkin lymphoma in Hodgkin Reed/Sternberg cells, is reduced or absent.

In embodiments relating to the use a MTA synergistic PRMT5 inhibitor for use in the treatment of cancer, the use may be indicated on the basis of results obtained from analysis of a sample obtained from the patient in need of treatment that indicates that the patient has a cancer that is wild type MTAP gene silenced. In such embodiments the determination of the wild type MTAP gene silenced may be made on the basis of a immunochemical assay for MTAP protein that reveals that MTAP protein expression in the nuclei and/or the cytoplasm of relevant tumour cells, for example in the case of Hodgkin lymphoma in Hodgkin Reed/Sternberg cells, is reduced or absent.

In embodiments relating to the use of a MTA synergistic PRMT5 inhibitor for the manufacture of a medicine for the treatment of cancer, the use of the resulting medicine may be indicated on the basis of the patient having been identified as having a wild type MTAP gene silenced tumour following analysis of a sample obtained from the patient. In such embodiments the determination of the wild type MTAP gene silenced may be made on the basis of a immunochemical assay for MTAP protein that reveals that MTAP protein expression in the nuclei and/or the cytoplasm of relevant tumour cells, for example in the case of Hodgkin lymphoma in Hodgkin Reed/Sternberg cells, is reduced or absent.

In embodiments, there is provided a kit comprising a MTA synergistic PRMT5 inhibitor and instructions for its use in the treatment of a wild type MTAP gene silenced cancer. In such embodiments, the instructions may characterise the wild type MTAP gene silenced cancer on the basis that the MTAP gene silencing
(a) delivers a partial or total loss of MTAP protein in relevant tumour cells; or
(b) delivers a partial or total loss of MTAP protein in the nucleus in relevant tumour cells; or
(c) leads to a reduction of MTAP protein expression in the nucleus of tumour cells; or
(d) leads to a reduction of MTAP protein expression in the nucleus of clonal tumour cells; or
(e) characteristically leads to accumulation of MTA in relevant tumour cells; or
(f) characteristically leads to accumulation of MTA in relevant tumour cells due MTAP gene silencing mediated by hypermethylation of MTAP; or
(g) characteristically leads to accumulation of MTA in relevant tumour cells due to epigenetic modification of the MTAP gene; or
(h) characteristically leads to accumulation of MTA in relevant tumour cells due to downregulation of MTAP at the protein level; or
(i) characteristically leads to accumulation of MTA in relevant tumour cells due to downregulation of MTAP protein expression; or
(j) results in epigenetically driven downregulation of MTAP mRNA; or
(k) leads to accumulation of MTA in relevant tumour cells due to partial or complete silencing of MTAP protein expression; or
(l) leads to accumulation of MTA in relevant tumour cells due to partial or complete silencing of MTAP protein expression due to epigenetic modification of the MTAP gene; or
(m) delivers a tumour that characteristically accumulates MTA due to partial or complete silencing of MTAP protein expression due to hypermethylation of the MTAP gene; or
(n) delivers a tumour that characteristically accumulates MTA due to downregulation of MTAP mRNA caused by hypermethylation at, or around, the MTAP gene; or
(o) delivers a tumour that characteristically accumulates MTA due to downregulation of MTAP mRNA caused by hypermethylation of the MTAP gene and/or an adjacent gene such as CDKN2A.

Thus, for illustration, the kit may provide instructions for the use of the MTA synergistic PRMT5 inhibitor in the treatment of a wild type MTAP gene silenced cancer that characteristically accumulates MTA due to MTAP gene silencing mediated by hypermethylation of MTAP gene as specified in the use (f).

In embodiments, the wild type MTAP gene silenced cancer is a cancer of the lymphatic system, for example a Hodgkin Lymphoma or a non-Hodgkin Lymphoma. In embodiments the wild type MTAP gene silenced cancer is a Hodgkin Lymphoma (HL) and may be a classical HL (cHL) categorised as nodular sclerosing (NSHL), mixed cellularity (MCHL), lymphocyte-rich (LRHL) and lymphocyte depleted (LDHL) or may be nodular lymphocyte-predominant HL. In embodiments the non-Hodgkin lymphoma is a Diffuse Large B-cell Lymphoma (DLBCL).

In embodiments, the wild type MTAP gene silenced cancer is a cancer selected from bladder cancer, breast cancer, kidney cancer, leukaemia, lung cancer, ovarian cancer, pancreatic, sarcoma or skin cancer.

In embodiments, the wild type MTAP gene silenced cancer is a Hodgkin Lymphoma.

In embodiments, the wild type MTAP gene silenced cancer is a Hodgkin Lymphoma and the determination of the gene silenced status is made on the basis of a immunohistochemical assay for MTAP protein that indicates that MTAP protein levels in the nuclei of Hodgkin Reed/Sternberg (HRS) cells are reduced or are null as assessed relative to normal cells such as the non HRS cells in the sample.

In embodiments, the MTA synergistic PRMT5 inhibitor is an inhibitor described in WO2021/163344.

In such embodiments the inhibitor has the general Formula I

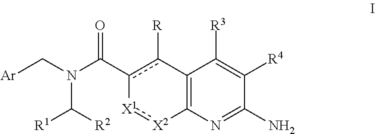

a tautomer thereof, a stereoisomer thereof, or a pharmaceutically acceptable salt of any of the foregoing,
wherein [symbol] represents a single or double bond;
x¹ and x² are both in each instance independently N or C; wherein if x¹ is C it can be optionally substituted with halo or C₁₋₆alkyl;
Ar is a six membered aromatic ring having 0-2 N atoms, wherein each Ar could be independently substituted with 0-2 R^{a} groups;
wherein R^{a} is in each instance independently selected from cyano, halo, optionally substituted C₁₋₆alkyl, C₁₋₆haloalkyl, OR\ NR^{c}R^{d}, —C(O) NR^{c}R^{d}, ═S, —SO₂, —SO₂C₁₋₆alkyl, —C(O)H, —C(O)C₁₋₆alkyl, C(O)OC₁₋₆alkyl, difluoro-pyrrolidinyl, and 4-to 6-membered heterocyclic ring, with 0-2 heteroatoms independently selected from O and N, and which heterocyclic ring can be further independently substituted with 0-2 halogen, C₁₋₆ alkyl, —C(O)H, —C(O)C₁₋₆alkyl or optionally substituted cycloalcoxyl;
wherein each R^{b} is in each instance independently selected from H, optionally substituted C₁₋₆ alkyl, wherein the substituents can be selected from halo; or oxetanyl;
wherein each R^{e} and R^{d} is independently selected from H, C₁₋₃alkyl, C₁₋₃ haloalkyl or —CO;
wherein R^{e} in each instance is selected from H or C₁₋₆alkyl;
wherein R^{f} and R^{g} in each instance is independently selected from H and C₁₋₆alkyl;
wherein R is H or methyl;
wherein R¹ and R² are in each instance is independently selected from H, optionally substituted C₁₋₆ alkyl, optionally substituted C₁₋₆alkynyl, —C(OR^{e}), optionally substituted single and double cyclyl having 0-3 N, S or O atoms; wherein the substituents are selected from halo, optionally substituted C₁₋₆alkyl, —C(O)NR^{f}R^{g}, OH and an optionally substituted 5-membered ring having 0-3 N atoms;
or R¹ and R² and the carbon atom to which they are attached can form an optionally substituted single or double carbocyclic or heterocyclic ring, which may be saturated, partially saturated or aromatic and further wherein the heterocyclic ring includes 1, 2 or 3 heteroatoms independently selected from N, O, and S;
wherein the substituents are selected from the group of optionally substituted C₁₋₆ alkyl, halo, CN, OR^{e} and —C(OR^{e}),
provided that R¹ and R² are not both H at the same time;
and wherein R³ and R⁴ are in each instance independently selected from H, halogen, alkynyl, cyano and C₁₋₆ alkyl, optionally substituted with halo or deuterium.

In such embodiments, the compound may be selected from the list of compounds presented in claim 19 of WO2021/163344 as presented at pages 267 to page 305 of the international publication.

In embodiments, the compound may be a compound of the Formula II below as claimed in claim 1 of WO2022/026892A1 and presented at page 2309 to page 2311 of the international publication. In such embodiments, the compound may be selected from the compounds presented in Table 1 of WO2022/026892A1 as presented at pages 122 to 470 of the international publication.

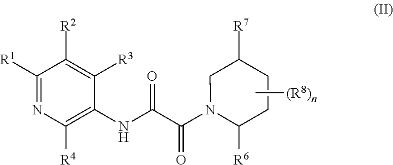

Within such embodiments, in embodiments the MTA synergistic PRMT5 inhibitor is N-(6-amino-5-methylpyridin-3-yl)-2-((2R,5S)-2-(benzo[d]thiazol-5-yl)-5-methylpiperidin-1-yl)-2-oxoacetamide:

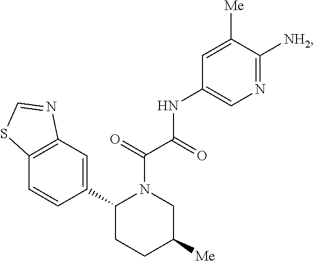 [SMILES: Cc1cc(NC(=O)C(=O)N2C[C@@H](C)CC[C@@H]2c2ccc3scnc3c2)cnc1N]

or a pharmaceutically acceptable salt thereof.

Within such embodiments, in embodiments the MTA synergistic PRMT5 inhibitor is N-(6-amino-5-methylpyridin-3-yl)-2-((2R,5S)-2-(benzo[d]thiazol-5-yl)-5-methylpiperidin-1-yl)-2-oxoacetamide:

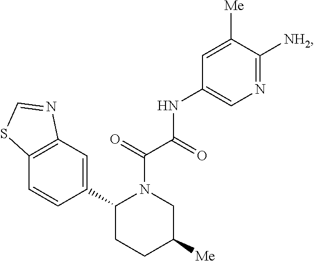 [SMILES: Cc1cc(NC(=O)C(=O)N2C[C@@H](C)CC[C@@H]2c2ccc3scnc3c2)cnc1N]

Within such embodiments, in embodiments the MTA synergistic PRMT5 inhibitor is a pharmaceutically acceptable salt of N-(6-amino-5-methylpyridin-3-yl)-2-((2R,5S)-2-(benzo[d]thiazol-5-yl)-5-methylpiperidin-1-yl)-2-oxoacetamide:

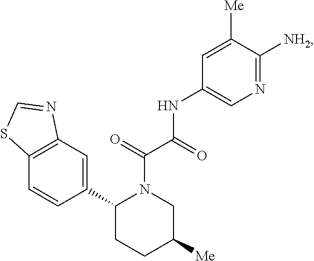 [SMILES: Cc1cc(NC(=O)C(=O)N2C[C@@H](C)CC[C@@H]2c2ccc3scnc3c2)cnc1N]

In embodiments, the MTA synergistic PRMT5 inhibitor is an inhibitor described in WO2022/115377A1. In such embodiments, the MTA synergistic PRMT5 inhibitor may be a compound of the Formula III below

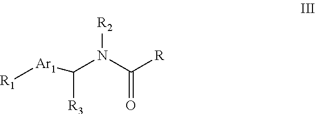

a tautomer thereof, a stereoisomer thereof, or a pharmaceutically acceptable salt of any of the foregoing, wherein:
R is a tricycle independently selected from the formulae IA and IB:

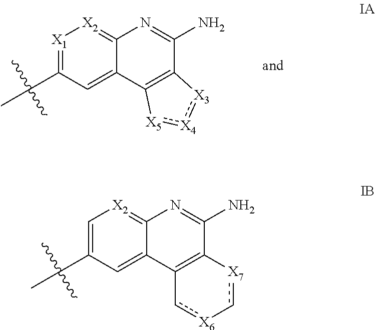

wherein [symbol] is a single or double bond,
x¹, x², x⁶ and x⁷ are in each instance N or C, wherein both x¹ and x² cannot be N at the same time, and wherein if x¹ is C, it can be optionally substituted with halo;
x³, x⁴ and x⁵ are at each instance independently selected from an optionally substituted C, O, N and S; wherein the substituents are independently selected from C₁₋₃ alkyl, C₁₋₃ alkyl (OH), wherein alkyl can be optionally substituted with halo;
R³ in each instance is independently selected from H or C₁₋₃ alkyl;
Ar¹ is a six membered optionally substituted aryl or heteroaryl independently selected from:

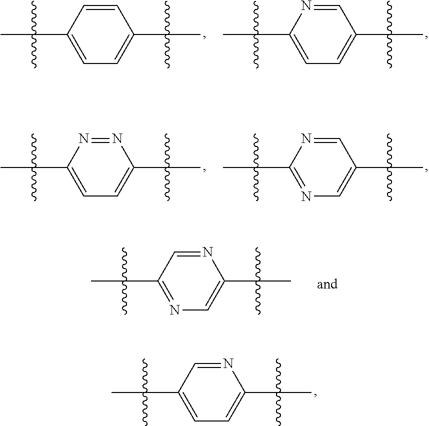 [SMILES: *c1ccc(*)cc1 ; *c1ccc(*)nc1 ; *c1ccc(*)nn1 ; *c1cnc(*)nc1 ; *c1cnc(*)cn1 ; *c1ccc(*)nc1]

wherein the substituents are independently selected from C₁₋₃ alkyl, —OC₁₋₃ alkyl or halo;
R¹ in each instance is independently selected from H, halo, optionally substituted C₁₋₃alkyl, wherein the substituents are selected from halo; —CN, optionally substituted —O—C₁₋₃alkyl, wherein the substituents are selected from halo; —C(O) OC₁₋₃ alkyl, wherein C₁₋₃alkyl can be optionally substituted with halo, and morpholinyl; and
R² in each instance is independently selected from an optionally substituted C₁₋₈ alkyl, wherein the substituents are selected from halo, hydroxy, amino, —O—C₁₋₃ alkyl or —CN; 5 or 6 membered cycle or heterocycle, optionally substituted with hydroxy, amino, an optionally substituted C₁₋₆alkyl, wherein the substituents are selected from halo; an optionally substituted C₁₋₆alkyl-O—C₁₋₃alkyl, wherein the substituents are selected from halo; 5,6,7,8-tetrahydro-[1,2,4]triazolo[1,5-a]pyridinyl; C₁₋₃alkyl-heterocyclyl, wherein the heterocyclyl is selected from optionally substituted 3,4-dihydro-2H-pyrano[2,3-c]pyridinyl; pyradazinyl, triazolyl, pyrimidinyl, tetrahydrofuranyl, 1H-pyrrolo[2,3-b]pyridinyl, cyclohexyl; wherein the substituents are selected from C₁₋₃ alkyl, —CN, and halo, or an optionally substituted C₁₋₆alkyl-O—C₁₋₃alkyl, wherein the substituents are selected from halo; optionally substituted phenyl, wherein the substituents are selected from halo or C₁₋₃alkyl.

In such embodiments, the compound may be selected from the compounds presented in claim 20 of WO2022/115377A1 as presented at pages 331 to 378 of the international publication. In such embodiments, in one embodiment the MTA synergistic PRMT5 inhibitor is selected from those presented in claim 21 of WO2022/115377 as presented on pages 377 and 378 of the international publication.

In embodiments, the MTA synergistic PRMT5 inhibitor is (P)-2-[4-[4-(aminomethyl)-1-oxo-2H-phthalazin-6-yl]-2-methyl-pyrazol-3-yl]-4-chloro-6-(cyclopropoxy)-3-fluoro-benzonitrile as described in Smith et al, https://doi.org/10.1016/i.bmc.2022.116947):

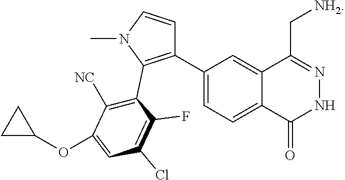

In embodiments, the MTA synergistic PRMT5 inhibitor is (P)-2-[4-[4-(aminomethyl)-1-oxo-2H-phthalazin-6-yl]-2-methyl-pyrazol-3-yl]-4-chloro-6-(cyclopropoxy)-3-fluoro-benzonitrile:

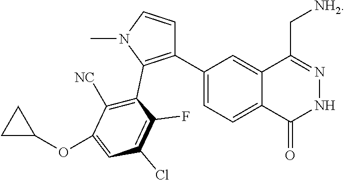

or a pharmaceutically acceptable salt thereof.

In embodiments, the MTA synergistic PRMT5 inhibitor is a pharmaceutically acceptable salt of (P)-2-[4-[4-(aminomethyl)-1-oxo-2H-phthalazin-6-yl]-2-methyl-pyrazol-3-yl]-4-chloro-6-(cyclopropoxy)-3-fluoro-benzonitrile:

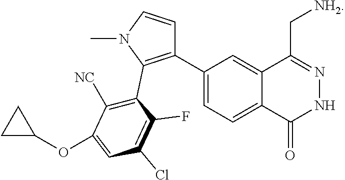

In embodiments, the MTA synergistic PRMT5 inhibitor (P)-2-[4-[4-(aminomethyl)-1-oxo-2H-phthalazin-6-yl]-2-methyl-pyrazol-3-yl]-4-chloro-6-(cyclopropoxy)-3-fluoro-benzonitrile hydrochloride:

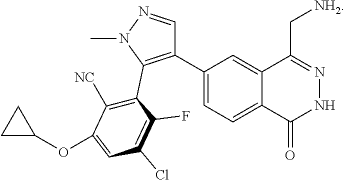

In embodiments the MTA synergistic PRMT5 inhibitor is a compound of Formula (IV) as described in WO2023/036974, or a pharmaceutically acceptable salt thereof:

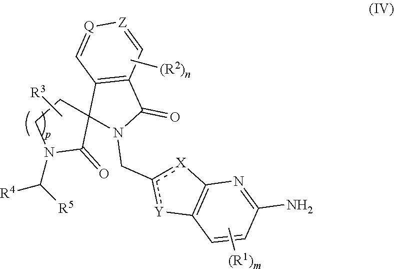

wherein:
the ring containing X and Y is a pyrrole and X is NH and Y is CH or X is CH and Y is NH;
Z is selected from CH, CF, CCl or, if Q is not N, N;
Q is selected from CH, CF, CCl or, if Z is not N, N;
m is 0, 1 or 2;
n is 0, 1 or 2;
p is 1 or 2;
R¹ is in each occurrence independently selected from F, Cl, CN, Me, CF₃, C₁-C₃ alkyl, cyclopropyl, C₁-C₃ fluoroalkyl, OMe or C₁-C₃ alkoxy;
R² is in each occurrence independently selected from F, Cl, Me, MeO and CF₃;
R³ is H, Me, C₁-C₃ alkyl or C₁-C₃ fluoroalkyl;
R⁴ is H, Me or C₁-C₃ alkyl;
R⁵ is H, Me, C₁-C₃ alkyl, C₁-C₃ fluoroalkyl, CH₂OMe, CH₂OCHF₂, CH₂OCF₃, CH₂O(C₁-C₃ alkyl), CH₂O(C₁-C₃ fluoroalkyl), C(CH₂CH₂) R⁶, CCR₇, CH₂R⁸, R⁹ or CH₂R¹⁰;
R⁶ is H, Me, CH₂F, CHF₂, CF₃, CH₂OH or CH₂OMe;
R⁷ is H, Me, cyclopropyl, C₁-C₃ alkyl, C₁-C₃ fluoroalkyl, C₃-C₆ cycloalkyl or a S-membered heteroaryl group optionally substituted with Me, C₁-C₃ alkyl, F or Cl;
R⁸ is a 5-membered heteroaryl optionally substituted with Me, C₁-C₃ alkyl, F or Cl;
R⁹ is an optionally substituted phenyl, 5-or 6-membered heteroaryl, or bicyclic heteroaryl group; and
R¹⁰ is an optionally substituted phenyl, 5-or 6-membered heteroaryl, or bicyclic heteroaryl group.

In embodiments, the MTA synergistic PRMT5 inhibitor is(S)-2-((5-Amino-6-fluoro-1H-pyrrolo[3,2-b]pyridin-2-yl)methyl)-5-fluoro-1′-(4-fluorobenzyl)spiro[isoindoline-1,3′-pyrrolidine]-2′,3-dione:

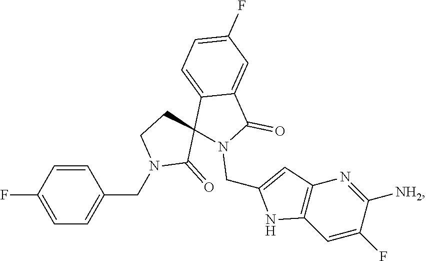 [SMILES: Nc1nc2cc(CN3C(=O)c4cc(F)ccc4[C@]34CCN(Cc3ccc(F)cc3)C4=O)[nH]c2cc1F]

or a pharmaceutically acceptable salt thereof.

In embodiments, the MTA synergistic PRMT5 inhibitor is(S)-2-((5-Amino-6-fluoro-1H-pyrrolo[3,2-b]pyridin-2-yl)methyl)-5-fluoro-1′-(4-fluorobenzyl)spiro[isoindoline-1,3′-pyrrolidine]-2′,3-dione:

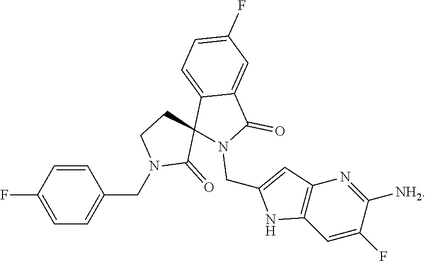 [SMILES: Nc1nc2cc(CN3C(=O)c4cc(F)ccc4[C@]34CCN(Cc3ccc(F)cc3)C4=O)[nH]c2cc1F]

In embodiments, the MTA synergistic PRMT5 inhibitor is a pharmaceutically acceptable salt of(S)-2-((5-Amino-6-fluoro-1H-pyrrolo[3,2-b]pyridin-2-yl)methyl)-5-fluoro-1′-(4-fluorobenzyl)spiro[isoindoline-1,3′-pyrrolidine]-2′,3-dione:

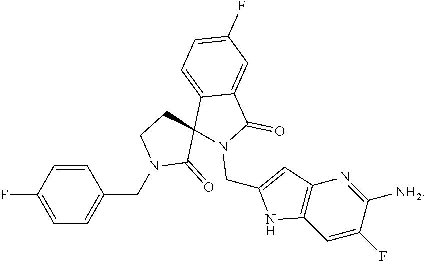 [SMILES: Nc1nc2cc(CN3C(=O)c4cc(F)ccc4[C@]34CCN(Cc3ccc(F)cc3)C4=O)[nH]c2cc1F]

In embodiments, the MTA synergistic PRMT5 inhibitor is(S)-2-((5-Amino-6-fluoro-1H-pyrrolo[3,2-b]pyridin-2-yl)methyl)-1′-(but-2-yn-1-yl)-5-fluorospiro[isoindoline-1,3′-pyrrolidine]-2′,3-dione:

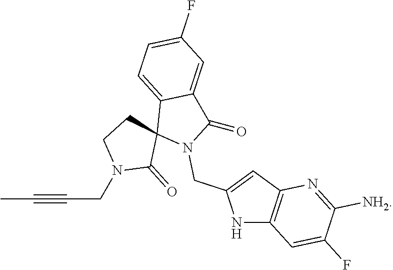 [SMILES: CC#CCN1CC[C@@]2(C1=O)c1ccc(F)cc1C(=O)N2Cc1cc2nc(N)c(F)cc2[nH]1]

or a pharmaceutically acceptable salt thereof.

In embodiments, the MTA synergistic PRMT5 inhibitor is(S)-2-((5-Amino-6-fluoro-1H-pyrrolo[3,2-b]pyridin-2-yl)methyl)-1′-(but-2-yn-1-yl)-5-fluorospiro[isoindoline-1,3′-pyrrolidine]-2′,3-dione:

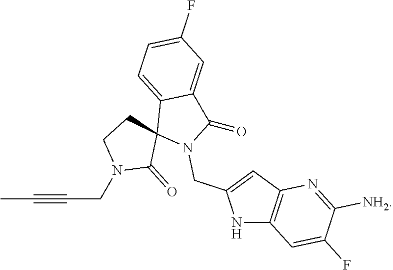 [SMILES: CC#CCN1CC[C@@]2(C1=O)c1ccc(F)cc1C(=O)N2Cc1cc2nc(N)c(F)cc2[nH]1]

In embodiments, the MTA synergistic PRMT5 inhibitor is a pharmaceutically acceptable salt of(S)-2-((5-Amino-6-fluoro-1H-pyrrolo[3,2-b]pyridin-2-yl)methyl)-1′-(but-2-yn-1-yl)-5-fluorospiro[isoindoline-1,3′-pyrrolidine]-2′,3-dione:

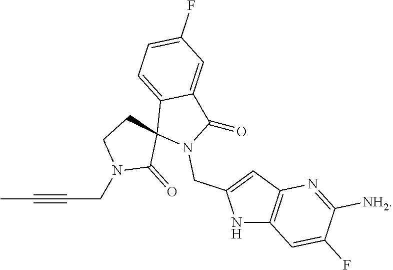 [SMILES: CC#CCN1CC[C@@]2(C1=O)c1ccc(F)cc1C(=O)N2Cc1cc2nc(N)c(F)cc2[nH]1]

In embodiments, the MTA synergistic PRMT5 inhibitor is an inhibitor of formula V as claimed in claim 1 of WO2021/050915 as presented on pages 321 and 322 of the international publication:

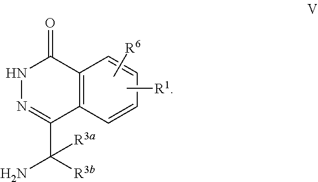

Within such embodiments, in embodiments the MTA synergistic PRMT5 inhibitor is selected from those claimed in claim 88 of WO2021/050915 as presented on pages 331 to 349 of the international publication.

Within such embodiments, in embodiments the MTA synergistic PRMT5 inhibitor is selected from those claimed in claim 89 of WO2021/050915 as presented on pages 349 and 350 of the international publication.

In embodiments, the MTA synergistic PRMT5 inhibitor is an inhibitor of formula VI-a, VI-b, VI-c, VI-d, VI-e or VI-f as claimed in claim 1 of WO2022/192745 as presented on pages 512 and 513 of the international publication:

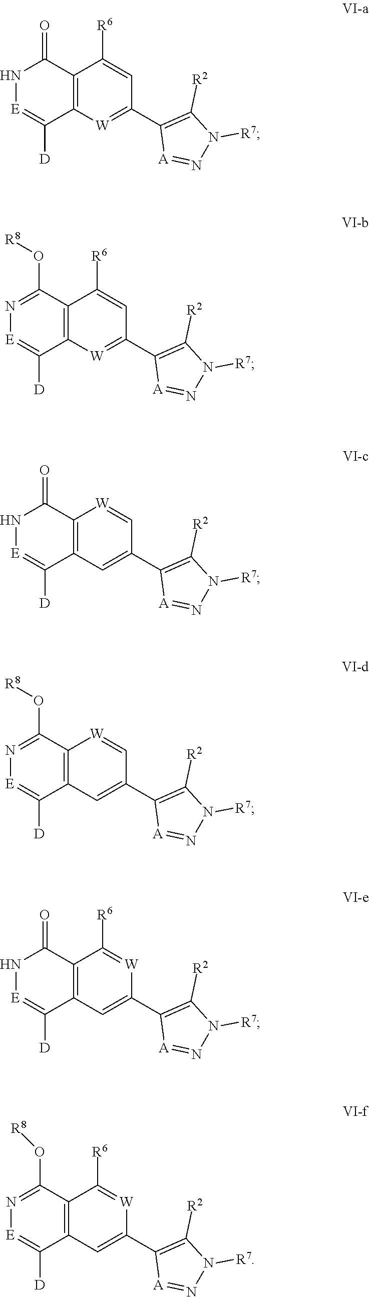

Within such embodiments, in embodiments the MTA synergistic PRMT5 inhibitor is selected from those claimed in claim 25 of WO2022/192745 as presented on pages 523 to 536 of the international publication.

Within such embodiments, in embodiments the MTA synergistic PRMT5 inhibitor is an inhibitor of formula VI-g, VI-h, VI-i, VI-j, VI-k, or VI-I as claimed in claim 26 of WO2022/192745 and as presented on pages 536 and 537 of the international publication:

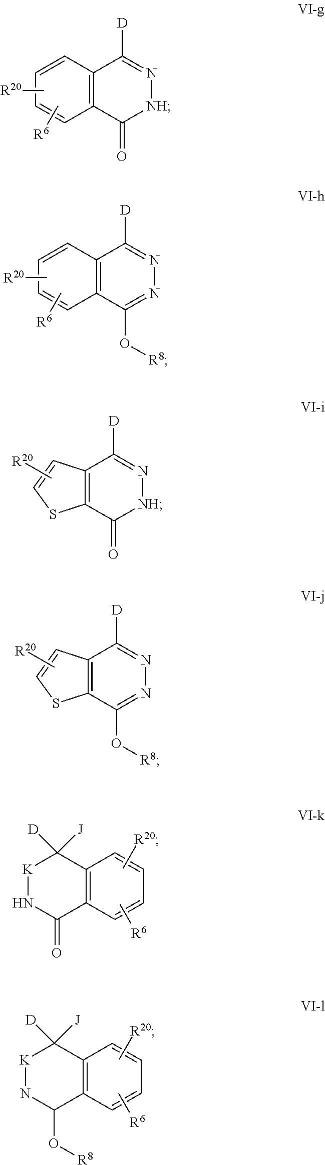

Within such embodiments, in embodiments, the MTA synergistic PRMT5 inhibitor is selected from those claimed in claim 27 of WO2022/192745 as presented on page 538 of the international publication.

In embodiments, the MTA synergistic PRMT5 inhibitor is an inhibitor of formula VII as claimed in claim 1 of WO2023/278564 as presented on pages 145 to 147 of the international publication:

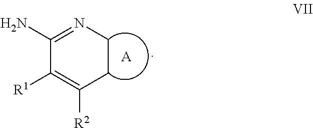

Within such embodiments, in embodiments the MTA synergistic PRMT5 inhibitor is selected from those claimed in claim 19 of WO2023/278564 as presented on pages 149 to 154 of the international publication.

In embodiments, the MTA synergistic PRMT5 inhibitor is an inhibitor of formula Vill as claimed in claim 1 of WO2022/132914 as presented on pages 188 and 189 of the international publication:

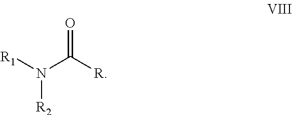

Within such embodiments, in embodiments the MTA synergistic PRMT5 inhibitor is selected from those claimed in claim 19 of WO2022/132914 as presented on pages 192 and 193 of the international publication.

Within such embodiments, in embodiments the MTA synergistic PRMT5 inhibitor is selected from those claimed in claim 20 of WO2022/132914 as presented on pages 194 and 195 of the international publication.

Within such embodiments, in embodiments the MTA synergistic PRMT5 inhibitor is (4-amino-1,3-dihydrofuro[3,4-c][1,7]naphthyridin-8-yl)-[(3S)-3-[4-(trifluoromethyl)phenyl]morpholin-4-yl]methanone:

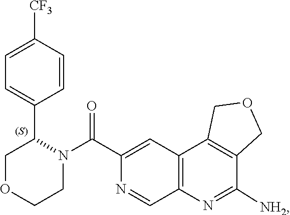

or a pharmaceutically acceptable salt thereof.

Within such embodiments, in embodiments the MTA synergistic PRMT5 inhibitor is (4-amino-1,3-dihydrofuro[3,4-c][1,7]naphthyridin-8-yl)-[(3S)-3-[4-(trifluoromethyl)phenyl]morpholin-4-yl]methanone:

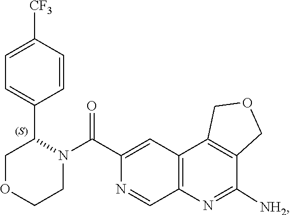

Within such embodiments, in embodiments the MTA synergistic PRMT5 inhibitor is a pharmaceutically acceptable salt of (4-amino-1,3-dihydrofuro[3,4-c][1,7]naphthyridin-8-yl)-[(3S)-3-[4-(trifluoromethyl)phenyl]morpholin-4-yl]methanone:

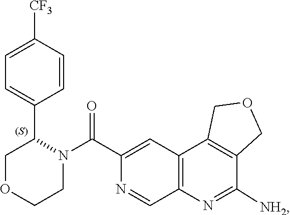

Within such embodiments, in embodiments the MTA synergistic PRMT5 inhibitor is (R)-(4-amino-1,3-dihydrofuro[3,4-c][1,7]naphthyridin-8-yl) (2-(4-(trifluoromethyl)phenyl) piperidin-1-yl) methanone:

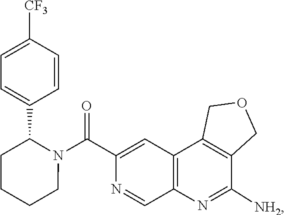

or a pharmaceutically acceptable salt thereof.

Within such embodiments, in embodiments the MTA synergistic PRMT5 inhibitor is (R)-(4-amino-1,3-dihydrofuro[3,4-c][1,7]naphthyridin-8-yl) (2-(4-(trifluoromethyl)phenyl) piperidin-1-yl) methanone:

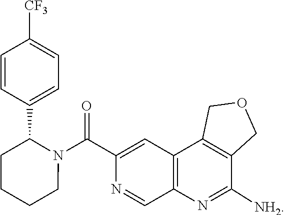

Within such embodiments, in embodiments the MTA synergistic PRMT5 inhibitor is a pharmaceutically acceptable salt of (R)-(4-amino-1,3-dihydrofuro[3,4-c][1,7]naphthyridin-8-yl) (2-(4-(trifluoromethyl)phenyl) piperidin-1-yl) methanone:

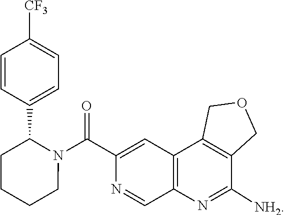

In embodiments, the MTA synergistic PRMT5 inhibitor is an inhibitor of formula IX as claimed in claim 1 of WO2022/169948 as presented on pages 240 and 241 of the international publication:

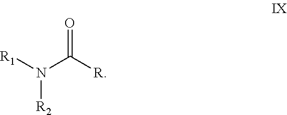

Within such embodiments, in embodiments the MTA synergistic PRMT5 inhibitor is selected from those claimed in claim 23 of WO2022/169948 as presented on pages 243 and 244 of the international publication.

In embodiments, the MTA synergistic PRMT5 inhibitor is an inhibitor of formula X as claimed in claim 1 of WO2023/081367 as presented on pages 161 and 162 of the international publication:

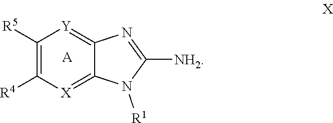

Within such embodiments, in embodiments the MTA synergistic PRMT5 inhibitor is an inhibitor of formula X-A as claimed in claim 6 of WO2023/081367 as presented on pages 164 and 165 of the international publication:

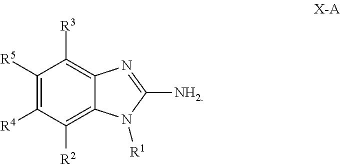

Within such embodiments, in embodiments the MTA synergistic PRMT5 inhibitor is selected from those claimed in claim 17 of WO2023/081367 as presented on pages 168 to 181 of the international publication.

Within such embodiments, in embodiments the MTA synergistic PRMT5 inhibitor is selected from those claimed in claim 18 of WO2023/081367 as presented on pages 181 to 185 of the international publication.

Within such embodiments, in embodiments the MTA synergistic PRMT5 inhibitor is selected from those claimed in claim 19 of WO2023/081367 as presented on pages 185 to 188 of the international publication.

Within such embodiments, in embodiments the MTA synergistic PRMT5 inhibitor is selected from those claimed in claim 20 of WO2023/081367 as presented on pages 188 to 189 of the international publication.

Within such embodiments, in embodiments the MTA synergistic PRMT5 inhibitor is selected from those claimed in claim 21 of WO2023/081367 as presented on pages 189 to 190 of the international publication.

In embodiments, the MTA synergistic PRMT5 inhibitor is an inhibitor of formula XI as claimed in claim 1 of CN116178347 as presented on page 2 of the A publication:

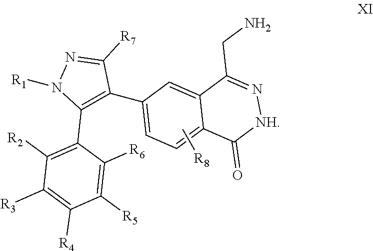

Within such embodiments, in embodiments the MTA synergistic PRMT5 inhibitor is selected from those claimed in claim 10 of CN116178347 as presented on pages 6 and 7 of the A publication.

In embodiments, the MTA synergistic PRMT5 inhibitor is an inhibitor of formula XII as claimed in claim 1 of WO2023/098439 as presented on pages 55 to 59 of the international publication:

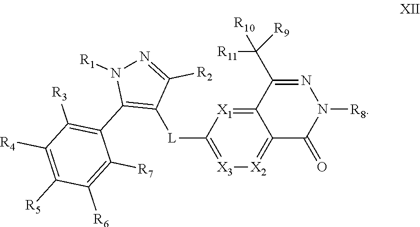

Within such embodiments, in embodiments the MTA synergistic PRMT5 inhibitor is selected from those claimed in claim 11 of WO2023/098439 as presented on pages 68 and 69 of the international publication.

In embodiments, the MTA synergistic PRMT5 inhibitor is an inhibitor of formula XIII as claimed in claim 1 of WO2021/086879 as presented on pages 497 and 498 of the international publication:

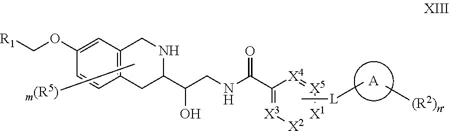

Within such embodiments, in embodiments the MTA synergistic PRMT5 inhibitor is an inhibitor of formula XIII-a as claimed in claim 5 of WO2021/086879 as presented on pages 499 and 500 of the international publication:

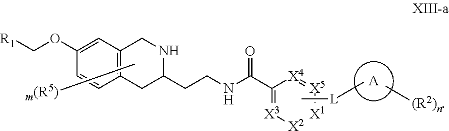

Within such embodiments, in embodiments the MTA synergistic PRMT5 inhibitor is an inhibitor of formula XIII-b as claimed in claim 63 of WO2021/086879 as presented on pages 507 to 509 of the international publication:

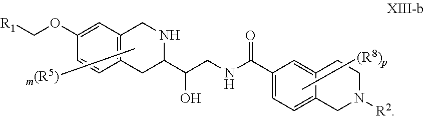

Within such embodiments, in embodiments the MTA synergistic PRMT5 inhibitor is an inhibitor of formula XIII-c as claimed in claim 65 of WO2021/086879 as presented on pages 509 and 510 of the international publication:

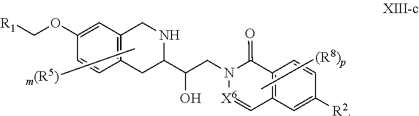

Within such embodiments, in embodiments the MTA synergistic PRMT5 inhibitor is selected from those recited in Table 1 of WO2021/086879 as presented on pages 103 to 114 of the international publication.

In embodiments, the MTA synergistic PRMT5 inhibitor is an inhibitor of formula XIV as claimed in claim 1 of CN116462676 as presented on pages 2 to 4 of the A publication:

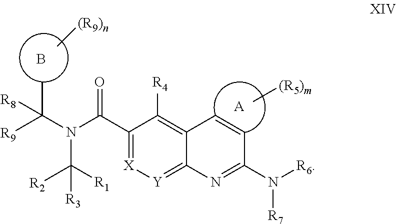

Within such embodiments, in embodiments the MTA synergistic PRMT5 inhibitor is selected from those claimed in claim 14 of CN116462676 as presented on pages 12 to 17 of the A publication.

In embodiments, the MTA synergistic PRMT5 inhibitor is an inhibitor of formula XV as claimed in claim 1 of CN116462677 as presented on pages 2 to 5 of the A publication:

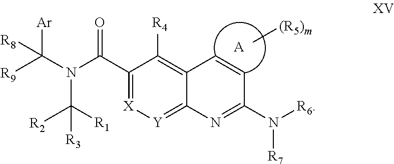

Within such embodiments, in embodiments the MTA synergistic PRMT5 inhibitor is selected from those claimed in claim 18 of CN116462677 as presented on pages 17 to 22 of the A publication.

In embodiments, the specification provides a pharmaceutical composition comprising a MTA synergistic PRMT5 inhibitor for use in the treatment of cancer, wherein the cancer is characterised as being wild type MTAP gene silenced. In such embodiments the MTA synergistic PRMT5 inhibitor may be selected from the list of inhibitors disclosed above.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range.

The term “pharmaceutical composition” refers to a preparation which is in such form as to permit the biological activity of the active ingredient, and which contains no additional components which are unacceptably toxic to a subject to which the composition would be administered. Such compositions can be sterile. A pharmaceutical composition according to the present specification will comprise an MTA synergistic PRMT5 inhibitor and at least one pharmaceutically acceptable excipient. The one or more pharmaceutically acceptable excipient(s) may be chosen from the group comprising fillers, binders, diluents and the like.

Terms such as “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent and/or slow the development of a targeted pathologic condition or disorder. Thus, those in need of treatment include those already with the disorder; those prone to have the disorder; and those in whom the disorder is to be prevented.

The term “subject” refers to a human that is to be the recipient of a particular treatment. Typically, the terms “subject” and “patient” are used interchangeably herein in reference to a human subject.

The MTA synergistic PRMT5 inhibitor, or a pharmaceutically acceptable salt thereof, will normally be administered via the oral route, in the form of pharmaceutical preparations comprising the active ingredient or a pharmaceutically acceptable salt or solvate thereof, or a solvate of such a salt, in a pharmaceutically acceptable dosage form. Depending upon the cancer and patient to be treated and the route of administration, the compositions may be administered at varying doses.

The pharmaceutical formulations of a MTA synergistic PRMT5 inhibitor may conveniently be administered in unit dosage form and may be prepared by any of the methods well-known in the pharmaceutical art, for example as described in Remington's Pharmaceutical Sciences, 17th ed., Mack Publishing Company, Easton, PA., (1985).

Pharmaceutical formulations suitable for oral administration may comprise one or more physiologically compatible carriers and/or excipients and may be in solid or liquid form. Tablets and capsules may be prepared with binding agents; fillers; lubricants; and surfactants. Liquid compositions may contain conventional additives such as suspending agents; emulsifying agents; and preservatives. Liquid compositions may be encapsulated in, for example, gelatin to provide a unit dosage form. Solid oral dosage forms include tablets, two-piece hard shell capsules and soft elastic gelatin (SEG) capsules.

Examples are provided below to facilitate appreciation of the therapeutic applicability of MTA synergistic inhibitors for use in the treatment of wild type MTAP gene silenced cancers.

### EXAMPLES

### Proliferation Assays

For the Hodgkin Lymphoma cell lines L428, L540, L1236, HDLM2 and KM-H2 (purchased from DSMZ, http://www.dsmz.de): Assay Ready Plates were prepared by adding compounds to 384-well plates (Corning #3712) using Echo Liquid Handler. On day 0, 1200 cells/well of L428, L540, or KMH2 cells in 60 μl of growth media (RPMI1640+10% FBS+1% L-Glu+1% P/S) were dispensed into the Assay Ready Plates, 10 μM as final top concentration, with 1:3 dilution, and 10 doses total. The same volume was also dispensed into 1 empty plate for Day 0 control plate. 30 μl/well of CellTiter Glo reagent (Promega #7573) was added into Day 0 plate, incubate at room temperature for 20 min in dark, Measure luminescence on plate reader Tecan M200 using 100 ms integration time, Oms attenuation and settle times. The assay plates were incubated for 7 days in incubator with 37° C., 90% humidity, 5% CO₂. Luminescence was measured as Day 0 control plate. IC₅₀ was calculated using GraphPad Prism 8 with Nonlinear regression (curve fit) analysis.

Alternatively, for HDLM2 (purchased from DSMZ): Cells were grown as adherent cells in growth media (phenol red free RPMI+10% FCS+2 mM glutamine). Cells were seeded into 96-well clear-bottomed black tissue-culture treated plates at 1000 cells/well in 90 μl growth media and placed in an incubator at 37° C., 5% CO₂. Compound was obtained in solution from the liquid bank at a concentration of 10 mM in DMSO then half-log serial dilutions performed in DMSO to make ×1000 fold stock solutions. Concentrations were then diluted 1 in 10 in DMSO followed by 1 in 10 in growth media to give an addition plate at x¹⁰ final concentration in 10% DMSO. 10 μl compound was then added to 90 μl cell (1 in 10 dilution) to give a concentration range of 0.1 nM to 10 μM in 1% DMSO. CellTiter Glo readings were taken at the point of dosing and at 6 days. Cell Titer Glo reagent was added to the volume of media currently on the cells (100 μl). Mix on plate shaker at room temperature for 2 minutes to induce cell lysis. Transfer 150 μl lysate to a white 96-well plate. Incubate at room temperature for 10 minutes to stabilise the luminescent signal (cover with silver plate seal). Read luminescence on Envision F (ultrasensitive luminescence 96-well protocol with 384 well aperture). IC₅₀ was calculated using GraphPad Prism 8 with Nonlinear regression (curve fit) analysis.

For the HCT116 isogenic cell line (the parental model purchased from ATCC, the MTAP KO clone generated in-house using CRISPR technology): Cells were harvested to a density of 400 cells per well (McCoys 5A+10% FCS+1% Glutamax), 40 μl/well seeded into 384-well plates (Greiner, Kremsmunster, Austria; 781090) using a Multidrop Combi. For Day 0 plates immediately add 4 μl Alamar Blue reagent (Thermo; DAL1100) using a multidrop combi and incubate for 3 h at 37° C., 5% CO₂. Day 0 cell plates were measured using Envision plate reader with fluorescence excitation wavelength of 540-570 nm (peak excitation is 570 nm), fluorescence emission at 580-610 nm (peak emission is 585 nm). Test compounds were added using an Echo 555 and placed in incubator at 37° C., 5% CO₂ and incubated for 4 more days. On Day 5, add 4 μl Alamar Blue reagent using a Multidrop Combi and incubate for 3 h at 37° C., 5% CO₂. Day 4 cell plates were measured using EnVision plate reader with fluorescence excitation wavelength of 540-570 nm, fluorescence emission at 580-610 nm. The rate of proliferation (IC₅₀ values) was determined using Genedata screener software by assessing the total cell number from the Envision plate reader for Day 0 and Day 4 plates.

### Western Blotting Experiments

Cell pellets were washed 2× ice-cold PBS and lysed in 1× SDS lysis buffer (100 mM Tris-HCl buffer, pH7.4, 10% Glycerol and 1% SDS), then frozen down at −80° C. Samples were thawed and samples heated at 95° C. for 5 minutes. After spinning at 14000 rpm for 10 minutes, the supernatant was transferred to fresh tubes. Protein concentration was measured using the Pierce™ BCA Protein Assay Kit (Pierce Cat #23225). 25 μg of protein from each cell line was loaded on NuPAGE™ 4-12% Bis-Tris gel (Invitrogen Cat #WG1403BOx10), run at 120V for 1.5 hr, and then transferred to the nitrocellulose membrane using the Bio Rad Semi-Dry transfer system (Bio Rad, model No. Trans-Blot SD Cell). The following primary and secondary antibodies were used to blot the membranes: MTAP (Cell Signaling, 4158), GAPDH (Cell Signaling, 2118), HRP-linked Anti-Rabbit IgG (Cell Signaling, 7074).

### Evaluation of MTAP Protein Expression in Hodgkin Lymphoma Tumour Samples by Immunohistochemistry

The IHC analysis was performed on the Ventana Benchmark platform (Roche Diagnostics) using the Ventana Human Immunohistochemical Staining Protocol as supplied by the instrument supplier.

Formalin-fixed paraffin embedded (FFPE) Hodgkin Lymphoma samples were obtained from Tristar Technology Group LLC, Washington DC USA.

Antigen retrieval was performed at: pH 8.55 at 100° C. degrees for 24 min.

Positive Controls: HCT116 cells (human MTAP wild-type colorectal cancer cell line), human tonsil cells (Tonsil FFPE block (ID 68282B2 (4)-4) commercially acquired from ProteoGenex Inglewood, CA 90301, USA).

Negative Controls: MCF7 cells (human MTAP deleted breast metastatic adenocarcinoma cell line), and xenograft tumours based on MCF7 MTAP null human breast cancer cells (FFPE block from AZ archival study bank).

The following equipment was used for performing the IHC analysis:
Ventana Benchmark Ultra and Prep Kit dispenser (Roche Diagnostics); Leica XL Autostainer (ST5010), and Leica CV5030 Coverslipper (leicabiosystems.com).

The following reagents were used:
Ventana Bulk Reagents: Benchmark Ultra LCS [Roche: 05424534001 (650-210)]; 10× EZ Prep Solution [Roche: 05279771001 (950-102)]; Reaction Buffer Concentrate 10× [Roche: 05353955001 (950-300)];
10×SSC [Roche: 05353947001 (950-110)]; ULTRA Cell Conditioning (ULTRA CC1) [Roche: 05424569001 (950-224)]

Ventana Dispenser Reagents: Optiview DAB IHC Detection Kit [Roche: 06396500001 (760-700)]; Hematoxylin [Roche: 05266726001 (760-2021)]; Bluing Reagent [Roche: 05266769001 (760-2037)]; Primary Antibody: MTAP (clone A8N9F) Rabbit IgG monoclonal antibody, CST #62765S (www.cellsignal.com)

### Staining Kit: OptiView DAB IHC Detection Kit #760-700 (Roche)

Additional Reagents: Deionised water, Fairy liquid (detergent, Proctor& Gamble).

### Procedure:

1. Refer to the user manual for the BenchMark ULTRA IHC/ISH System (Roche) for how to operate the Ventana and fill bulk reagents, empty waste containers and print slide labels. Note. EZ prep solution and reaction buffer are diluted to 1× in deionised water before use.

2. In a Ventana Prep Kit dispenser, prepare MTAP (A8N9F) Rabbit mAb [CST #62765S] to a working concentration of 0.5 μg/ml in Ventana Diluent [Roche: 05261899001 (251-018)] OR 2 μg/ml in Ventana Diluent with Casein [Roche: 06440002001 (760-219)].

3. In a Ventana Option dispenser, register and fill with Dako Serum Free Protein Block [Agilent: X090930-2].

4. Print labels for the slides (refer to user manual for the BenchMark ULTRA IHC/ISH System).

5. Apply the labels to the slides and load the slides and reagent dispensers onto the Ventana, then set the machine running.

6. Upon completion of Ventana run; remove slides from slide trays and load into a coverslipper rack.

7. Wash the slides in soapy water to remove LCS oil and then rinse in running tap water.

8. Repeat the wash then load rack into Leica XL autostainer and select program 3 ‘Ventana Clearing’ to pass slides through running water, graded ethanols and xylene.

9. Once finished coverslip the slides on the CV5030 coverslipper using 24×50 mm coverslips.

10. Scan at 40× on the Leica Aperio AT2 scanner (https://www.leicabiosystems.com/) prior to manual pathology scoring. Manual scoring was conducted by an experienced pathologist. H-scoring was adapted and used for nuclear MTAP staining in tumour cells. Presence and intensity of any cytoplasmic MTAP staining in tumour cells was also determined and noted for each sample. For a description of H Scoring and its application see: D. A. Budwit-Novotny et al Cancer Res. 1986; 46:5419-5425, F. Aeffner et al Archives of pathology & laboratory medicine 141 (9), 1267-1275, D. K. Meyerholz et al, Laboratory Investigation 98 (7): 844-855.

### Preparation of(S)-2-((5-Amino-6-fluoro-1H-pyrrolo[3,2-b]pyridin-2-yl)methyl)-5-fluoro-1′-(4-fluorobenzyl)spiro[isoindoline-1,3′-pyrrolidine]-2′,3-dione (Compound A)

Compound A may be prepared according to the methods disclosed in WO2023/036974, such as the methods disclosed herein.

### Methyl 2-(2-bromo-4-fluorophenyl)acetate

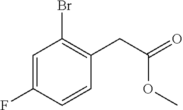

Thionyl chloride (31.3 mL, 429.1 mmol) was added dropwise carefully to 2-(2-bromo-4-fluorophenyl) acetic acid (CAS No. 61150-59-2) (100 g, 429.1 mmol) in MeOH (400 mL) at rt. The reaction mixture was stirred at 60° C. for 4 hours, cooled and the solvent was removed in vacuo. The residue was partitioned between EtOAc (250 mL) and saturated NaHCO₃ (200 mL). The organic phase was washed with water (100 mL), brine (100 mL), passed through a phase separating filter paper and the solvent was removed in vacuo to afford the title compound (105 g, 99%) as a colourless oil. ¹H NMR (400 MHZ, DMSO-d6, 30° C.) 3.64 (3H, s), 3.83 (2H, s), 7.25 (1H, td), 7.48 (1H, dd), 7.58 (1H, dd)); m/z MH⁺ not observed.

### Methyl 5-fluoro-2-(2-methoxy-2-oxoethyl)benzoate

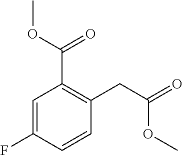 [SMILES: COC(=O)Cc1ccc(F)cc1C(=O)OC]

Methyl 2-(2-bromo-4-fluorophenyl)acetate (45.0 g, 182.14 mmol) and triethylamine (27.90 mL, 200.35 mmol) were placed in a steel pressure vessel with MeOH (300 ml). [1,1′-Bis(diphenylphosphino) ferrocene]dichloropalladium (II) (complex with dichloromethane) (4.46 g, 5.46 mmol) was added and the vessel was sealed. The vessel was purged with carbon monoxide and then charged to 7 bar with carbon monoxide. The pressure vessel was heated to 100° C. and stirred for 2 hours. The reaction mixture was allowed to cool, vented and filtered to remove catalyst. The solvent was removed in vacuo and the residue was dissolved in EtOAc (250 mL), washed with water (2×200 mL) and brine (100 mL). The organic phase was passed through a phase separating filter paper and the solvent was removed in vacuo. The crude product was purified by flash silica chromatography, elution gradient 0 to 50% EtOAc in heptane. Pure fractions were evaporated to dryness to afford the title compound (38.40 g, 93%) as a pale yellow oil. ¹H NMR (400 MHZ, DMSO-d6, 30° C.) 3.60 (3H, s), 3.80 (3H, s), 3.99 (2H, s), 7.42-7.49 (2H, m), 7.66 (1H, ddd); m/z MH⁺227.

### rac-Methyl-2-(1-bromo-2-methoxy-2-oxoethyl)-5-fluorobenzoate

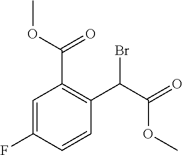 [SMILES: COC(=O)c1cc(F)ccc1C(Br)C(=O)OC]

Methyl 5-fluoro-2-(2-methoxy-2-oxoethyl)benzoate (47.0 g, 207.8 mmol) was dissolved in chloroform (450 mL). 1-Bromopyrrolidine-2,5-dione (55.5 g, 311 mmol) was added followed by 2,2′-azobis(2-methylpropionitrile) (3.41 g, 20.8 mmol) and the reaction mixture was stirred at reflux for 72 hours. The reaction mixture was cooled and washed with water (2×250 mL), brine (100 mL), passed through a phase separating filter paper and the solvent was removed in vacuo. The crude product was purified by flash silica chromatography, elution gradient 0 to 40% EtOAc in heptane. Pure fractions were evaporated to dryness to afford the title compound (50.50 g, 80%) as a colourless oil. ¹H NMR (400 MHz, DMSO-d6, 30° C.) 3.71 (3H, s), 3.86 (3H, s), 6.51 (1H, s), 7.56 (1H, td), 7.66 (1H, dd), 7.81 (1H, dd); m/z MH⁺ not observed.

### rac-Methyl 5-fluoro-2-(4-methoxybenzyl)-3-oxoisoindoline-1-carboxylate

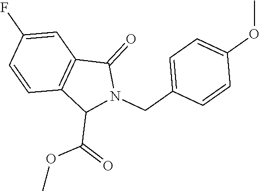 [SMILES: COC(=O)C1c2ccc(F)cc2C(=O)N1Cc1ccc(OC)cc1]

4-Methoxybenzylamine (23.5 g, 171 mmol) was placed in a flask with MeCN (300 mL) and sodium bicarbonate (23.9 g, 285 mmol) was added. rac-Methyl 2-(1-bromo-2-methoxy-2-oxoethyl)-5-fluorobenzoate (43.5 g, 142 mmol), dissolved in MeCN (100 mL), was added slowly via dropping funnel as the reaction mixture was brought up to 80° C. The reaction mixture was stirred at 80° C. for 3 hours. The reaction mixture was allowed to cool, most of the MeCN was removed in vacuo and the residue was partitioned between EtOAc (400 mL) and water (400 mL). The aqueous phase was re-extracted with EtOAc (100 mL), the organics were combined and washed with brine (50 mL). The organic phase was passed through a phase separating filter paper and the solvent was removed in vacuo. The crude product was purified by flash silica chromatography, elution gradient 0 to 50% EtOAc in heptane. Pure fractions were evaporated to dryness to afford the title compound (45.3 g, 96%) as a pale yellow oil. ¹H NMR (400 MHZ, DMSO-d6, 30° C.) 3.69 (3H, s), 3.73 (3H, s), 4.31 (1H, d), 5.04 (1H, d), 5.18 (1H, s), 6.87-6.94 (2H, m), 7.17-7.24 (2H, m), 7.50 (1H, ddd), 7.57 (1H, dd), 7.62 (1H, dd); m/z MH⁺330.

### rac-Methyl 1-allyl-5-fluoro-2-(4-methoxybenzyl)-3-oxoisoindoline-1-carboxylate

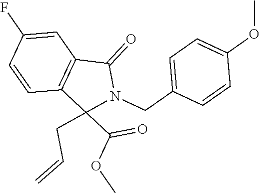 [SMILES: C=CCC1(C(=O)OC)c2ccc(F)cc2C(=O)N1Cc1ccc(OC)cc1]

rac-Methyl 5-fluoro-2-(4-methoxybenzyl)-3-oxoisoindoline-1-carboxylate (24.0 g, 72.9 mmol), allyl acetate (11.8 mL, 109 mmol), tris(dibenzylideneacetone) dipalladium (0) (1.67 g, 1.82 mmol) and N,N′-((1R,2R)-cyclohexane-1,2-diyl)bis(2-(diphenylphosphaneyl)benzamide) (2.52 g, 3.64 mmol) were stirred in THF (400 mL) at 5° C. under nitrogen. 1,1,3,3-tetramethylguanidine (13.7 mL, 109 mmol) was then added dropwise. The reaction mixture was stirred at 5° C. for 5 minutes. The THF was removed in vacuo. The reaction mixture was partitioned between EtOAc (400 mL) and water (400 mL) and the organic phase was passed through a phase separating filter paper. The solvent was removed in vacuo to afford an orange oil. The crude product was purified by flash silica chromatography, elution gradient 0 to 50% EtOAc in heptane. Pure fractions were evaporated to dryness to afford the title compound (25.8 g, 96%) as a cream solid. ¹H NMR (400 MHZ, DMSO-d6, 30° C.) 3.04-3.20 (2H, m), 3.26 (3H, s), 3.73 (3H, s), 4.52 (1H, d), 4.71 (1H, d), 4.74-4.94 (3H, m), 6.82-6.96 (2H, m), 7.28-7.39 (2H, m), 7.45-7.58 (2H, m), 7.63 (1H, dd); m/z MH⁺370.

### Methyl(S)-1-allyl-5-fluoro-2-(4-methoxybenzyl)-3-oxoisoindoline-1-carboxylate

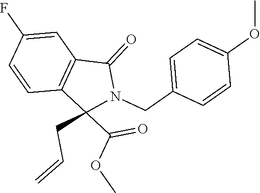 [SMILES: C=CC[C@@]1(C(=O)OC)c2ccc(F)cc2C(=O)N1Cc1ccc(OC)cc1]

rac-Methyl 1-allyl-5-fluoro-2-(4-methoxybenzyl)-3-oxoisoindoline-1-carboxylate (˜70:30 in favour of the desired(S) enantiomer) (25.8 g, 69.7 mmol) was purified by SFC chromatography (Column: Phenomenex C1, 30×250 mm, 5 micron, mobile phase: 10% IPA+0.1% DEA/90% scCO₂, flow rate: 90 ml/min, BPR: 120 bar, column temperature: 40° C., UV max 210 nm). Pure fractions were evaporated to dryness to afford the title compound (15.1 g, 56%) as a as a white solid. ¹H NMR (400 MHZ, DMSO-d6, 30° C.) 3.04-3.20 (2H, m), 3.26 (3H, s), 3.73 (3H, s), 4.52 (1H, d), 4.71 (1H, d), 4.74-4.94 (3H, m), 6.82-6.96 (2H, m), 7.28-7.39 (2H, m), 7.45-7.58 (2H, m), 7.63 (1H, dd); m/z MH⁺370.

### Methyl(S)-5-fluoro-2-(4-methoxybenzyl)-3-oxo-1-(2-oxoethyl) isoindoline-1-carboxylate

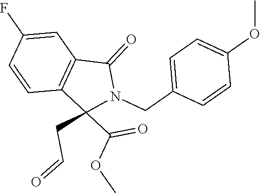 [SMILES: COC(=O)[C@]1(CC=O)c2ccc(F)cc2C(=O)N1Cc1ccc(OC)cc1]

To a solution of methyl(S)-1-allyl-5-fluoro-2-(4-methoxybenzyl)-3-oxoisoindoline-1-carboxylate (60.0 g, 162 mmol) in 1,4-dioxane (800 mL) and water (200 mL) was added osmium (VIII) oxide (4% in water) (5.16 mL, 0.81 mmol), sodium periodate (87.0 g, 406 mmol) and 2,6-dimethylpyridine (37.8 mL, 324 mmol). The reaction mixture was stirred at rt for 18 hours. The reaction mixture was filtered to remove salts and rinsed through with dichloromethane (DCM, 500 mL). The filtrate was placed in a separating funnel with water (500 mL) and partitioned. The aqueous phase was re-extracted with DCM (300 mL), the organic phases were combined, passed through a phase separating filter paper and the solvent was removed in vacuo. The crude product was purified by flash silica chromatography, elution gradient 0 to 50% EtOAc in heptane. Pure fractions were evaporated to dryness to afford the title compound (50.1 g, 83%) as a white crystalline solid. ¹H NMR (400 MHZ, DMSO-d6, 30° C.) 3.40 (3H, s), 3.42-3.56 (2H, m), 3.72 (3H, s), 4.58 (1H, d), 4.74 (1H, d), 6.80-6.92 (2H, m), 7.19-7.27 (2H, m), 7.52 (1H, ddd), 7.60 (1H, dd), 7.69 (1H, dd), 9.07 (1H, t); m/z MH⁺372.

### (S)-5-Fluoro-1′-(4-fluorobenzyl)-2-(4-methoxybenzyl)spiro[isoindoline-1,3′-pyrrolidine]-2′,3-dione

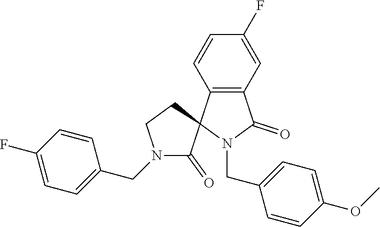 [SMILES: COc1ccc(CN2C(=O)c3cc(F)ccc3[C@]23CCN(Cc2ccc(F)cc2)C3=O)cc1]

Methyl(S)-5-fluoro-2-(4-methoxybenzyl)-3-oxo-1-(2-oxoethyl) isoindoline-1-carboxylate (45 g, 121.2 mmol) and 4-fluorobenzylamine (22.75 g, 181.8 mmol) were placed in a flask with 1,2-dichloroethane (600 mL) and stirred for 1 hour. The reaction mixture was placed in an ice bath and acetic acid (13.87 mL, 242.4 mmol) was added followed by sodium triacetoxyborohydride (51.4 g, 242.4 mmol). The reaction mixture was stirred at rt for 18 hours. The reaction mixture was neutralised with 2M NaOH, diluted with water (200 mL), and extracted with DCM (2×200 mL). The combined organic phases were passed through a phase separating filter paper and the solvent was removed in vacuo to afford the title compound as a pale yellow oil. Used crude in the next reaction assuming 100% yield. m/z MH⁺449.

### Step 2; (S)-5-Fluoro-1′-(4-fluorobenzyl)spiro[isoindoline-1,3′-pyrrolidine]-2′,3-dione

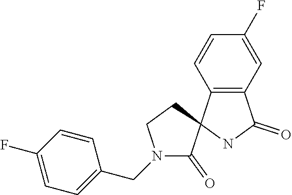 [SMILES: O=C1N[C@@]2(CCN(Cc3ccc(F)cc3)C2=O)c2ccc(F)cc21]

(S)-5-Fluoro-1′-(4-fluorobenzyl)-2-(4-methoxybenzyl)spiro[isoindoline-1,3′-pyrrolidine]-2′,3-dione (54.30 g, 121.08 mmol) was placed in a flask with MeCN (500 mL) and water (250 mL). Ammonium cerium (IV) nitrate (199.0 g, 363.2 mmol) was added and the reaction mixture was stirred at rt for 1 hour. The reaction mixture was partitioned between DCM (500 mL) and water (500 mL). The organic phase was washed with water (200 mL) and brine (200 mL), passed through a phase separating filter paper and the solvent was removed in vacuo. The crude product was purified by flash silica chromatography, elution gradient 0 to 100% (10% MeOH in EtOAc) in heptane. Pure fractions were evaporated to dryness to afford the title compound (30.50 g, 77%) as a cream solid. ¹H NMR (400 MHZ, DMSO-d6, 30° C.) 2.37-2.44 (1H, m), 2.45-2.49 (1H, m), 3.48 (1H, ddd), 3.60 (1H, dt), 4.49 (2H, s), 7.19-7.25 (2H, m), 7.32-7.37 (2H, m), 7.46 (3H, d), 9.11 (1H, s); m/z MH⁺329.

### Step 3; (S)-2-((5-Chloro-6-fluoro-1-((2-(trimethylsilyl) ethoxy)methyl)-1H-pyrrolo[3,2-b]pyridin-2-yl)methyl)-5-fluoro-1′-(4-fluorobenzyl)spiro[isoindoline-1,3′-pyrrolidine]-2′,3-dione

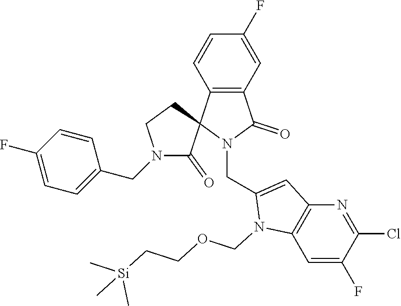 [SMILES: C[Si](C)(C)CCOCn1c(CN2C(=O)c3cc(F)ccc3[C@]23CCN(Cc2ccc(F)cc2)C3=O)cc2nc(Cl)c(F)cc21]

(S)-5-Fluoro-1′-(4-fluorobenzyl)spiro[isoindoline-1,3′-pyrrolidine]-2′,3-dione (27 g, 82.24 mmol) and 5-chloro-2-(chloromethyl)-6-fluoro-1-((2-(trimethylsilyl) ethoxy)methyl)-1H-pyrrolo[3,2-b]pyridine (30.20 g, 86.35 mmol) were placed in a flask with dry dimethylformamide (DMF, 120 mL). Caesium carbonate (67.00 g, 205.6 mmol) was added and the reaction mixture was stirred at 50° C. for 1 hour.

The reaction mixture was partitioned between water (500 mL) and EtOAc (500 mL) and the aqueous phase was re-extracted with EtOAc (250 mL). The organic phases were combined, washed with water (3×250 mL), brine (200 mL), passed through a phase separating filter paper and the solvent was removed in vacuo. The residue was triturated with diethyl ether (200 mL) and the resulting solid was filtered, washed with ether and dried to afford the title compound (42.20 g, 80%) as a cream solid. 1H NMR (400 MHZ, DMSO-d6, 30° C.)−0.13 (9H, s), 0.55-0.80 (2H, m), 2.32-2.41 (1H, m), 2.52 (1H, d), 3.30-3.38 (1H, m), 3.41-3.51 (2H, m), 3.57-3.69 (1H, m), 4.22-4.36 (2H, m), 4.75 (1H, d), 5.11 (1H, d), 5.53 (1H, d), 5.61 (1H, d), 6.53 (1H, s), 7.13-7.23 (4H, m), 7.45-7.54 (2H, m), 7.57-7.64 (1H, m), 8.28 (1H, dd); m/z MH⁺641.

### (S)-2-((5-((Diphenylmethylene)amino)-6-fluoro-1-((2-(trimethylsilyl) ethoxy)methyl)-1H-pyrrolo [3,2-b]pyridin-2-yl)methyl)-5-fluoro-1′-(4-fluorobenzyl)spiro[isoindoline-1,3′-pyrrolidine]-2′,3-dione

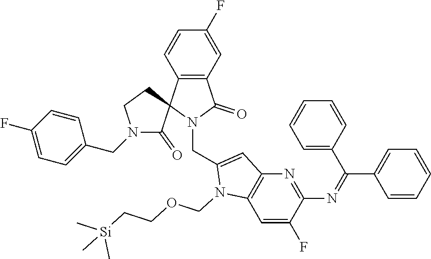 [SMILES: C[Si](C)(C)CCOCn1c(CN2C(=O)c3cc(F)ccc3[C@]23CCN(Cc2ccc(F)cc2)C3=O)cc2nc(N=C(c3ccccc3)c3ccccc3)c(F)cc21]

(S)-2-((5-Chloro-6-fluoro-1-((2-(trimethylsilyl) ethoxy)methyl)-1H-pyrrolo[3,2-b]pyridin-2-yl)methyl)-5-fluoro-1′-(4-fluorobenzyl)spiro[isoindoline-1,3′-pyrrolidine]-2′,3-dione (41.80 g, 65.19 mmol), diphenylmethanimine (14.18 g, 78.23 mmol) and sodium 2-methylpropan-2-olate (12.53 g, 130.4 mmol) were placed in a flask with toluene (300 mL) and the reaction mixture was degassed by bubbling nitrogen through the mixture for 10 minutes. tBuXPhos (2.77 g, 6.52 mmol) and tris(dibenzylideneacetone) dipalladium (0) (2.99 g, 3.26 mmol) were added and the reaction mixture was then stirred at 65° C. for 30 minutes. The reaction mixture was allowed to cool and partitioned between EtOAc (600 mL) and water (600 mL). The organic phase was washed with brine (200 mL), passed through a phase separating filter paper and the solvent was removed in vacuo. The crude product was purified by flash silica chromatography, elution gradient 0 to 100% EtOAc in heptane. Pure fractions were evaporated to dryness to afford the title compound (49.50 g, 97%) as a yellow solid. ¹H NMR (400 MHZ, DMSO-d6, 30° C.)−0.15 (9H, s), 0.57-0.76 (2H, m), 2.29-2.39 (1H, m), 2.39-2.48 (1H, m), 3.26-3.29 (1H, m), 3.34-3.45 (2H, m), 3.53-3.66 (1H, m), 4.24 (2H, s), 4.67 (1H, d), 5.04 (1H, d), 5.44 (2H, q), 6.32 (1H, s), 7.11 (2H, dd), 7.17-7.26 (7H, m), 7.43-7.54 (4H, m), 7.55-7.61 (2H, m), 7.68-7.76 (2H, m), 7.81 (1H, d); m/z MH⁺786.

### (S)-2-((5-Amino-6-fluoro-1H-pyrrolo[3,2-b]pyridin-2-yl)methyl)-5-fluoro-1′-(4-fluorobenzyl)spiro[isoindoline-1,3′-pyrrolidine]-2′,3-dione

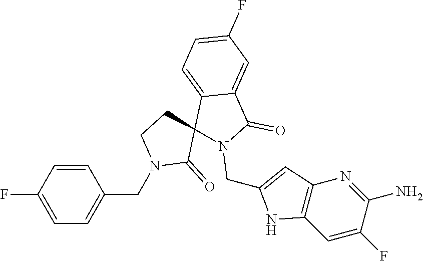 [SMILES: Nc1nc2cc(CN3C(=O)c4cc(F)ccc4[C@]34CCN(Cc3ccc(F)cc3)C4=O)[nH]c2cc1F]

(S)-2-((5-((Diphenylmethylene)amino)-6-fluoro-1-((2-(trimethylsilyl) ethoxy)methyl)-1H-pyrrolo[3,2-b]pyridin-2-yl)methyl)-5-fluoro-1′-(4-fluorobenzyl)spiro[isoindoline-1,3′-pyrrolidine]-2′,3-dione (49.50 g, 62.98 mmol) was placed in a flask with 2,2,2-trifluoroacetic acid (96 mL, 1259.63 mmol). 0.50 ml of water was added and the reaction mixture was stirred at 40° C. for 4 hours. The 2,2,2-trifluoroacetic acid was removed in vacuo and the residue was dissolved in MeCN (75 mL). Ammonium hydroxide (28-30% in water) (73.60 mL, 1889.45 mmol) was added and the reaction mixture was stirred at 40° C. for 4 hours and then at rt overnight. The resulting solid was filtered off and washed with MeCN (100 mL) to afford ˜20 g of the desired compound. The filtrate was reduced to ˜ 200 mL and purified by reverse phase chromatography (Interchim C18-HP Flash column, 2×415 g, 100 ml loading of solution/run), using decreasingly polar mixtures of water (containing by volume 1% NH₄OH (28-30% in H₂O)) and MeCN as eluents (30-60% gradient). Fractions containing the desired compound were combined and the previous solid (˜20 g) obtained was added. The slurry was stirred for 1 hour and then the MeCN was removed in vacuo resulting in the formation of a pale yellow precipitate. The solid was filtered off and dried under vacuum for 2 hours. The solid was then suspended in MeCN (150 mL) and the slurry was gently refluxed for 2 hours before allowing to cool overnight. The solid was filtered off and dried under vacuum to afford the title compound (19.54 g, 63%) as a cream crystalline solid. ¹H NMR (400 MHZ, DMSO-d6, 30° C.) 2.34-2.40 (2H, m), 3.36 (1H, ddd), 3.60 (1H, dt), 4.29 (1H, d), 4.39-4.52 (2H, m), 5.03 (1H, d), 5.48 (2H, s), 6.02 (1H, d), 7.18-7.27 (2H, m), 7.27-7.39 (3H, m), 7.46-7.55 (2H, m), 7.59 (1H, ddd), 10.69 (1H, d); m/z MH⁺492.

### Preparation of(S)-2-((5-Amino-6-fluoro-1H-pyrrolo[3,2-b]pyridin-2-yl)methyl)-1′-(but-2-yn-1-yl)-5-fluorospiro[isoindoline-1,3′-pyrrolidine]-2′,3-dione (Compound C)

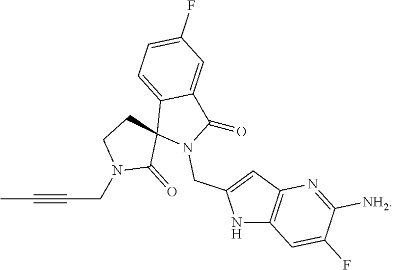 [SMILES: CC#CCN1CC[C@@]2(C1=O)c1ccc(F)cc1C(=O)N2Cc1cc2nc(N)c(F)cc2[nH]1]

Compound C was prepared according to the methods disclosed in WO2023/036974.

### Biological Example 1: Assessment of the Effect of Compound C Treatment on In Vivo Tumor Growth Inhibition and Target Engagement in L540 HL Xenograft Model

This study was performed to evaluate tumor growth inhibition and pharmacodynamic changes following treatment with the MTA synergistic PRMT5 inhibitor Compound C in the MTAP silenced L540 HL xenograft model. Three doses of Compound C (Dose Level 1, Dose Level 2 and Dose Level 3) were tested. Pharmacodynamic changes were assessed by western blot for a decrease in SDMA (marker of target engagement) following treatment with Compound C.

TABLE 4
Test animals used in the study

| | |
|---|---|
| Species | Mouse (Mus Musculus) |
| Strain | NOD/SCID |
| Source | GemPharmatech Co., Ltd |
| Total number | 40 females |
| Age at start of study | 6-8 weeks old |
| Body weight range | +18 g |
| Identification | Ear tag |

TABLE 5
Cell line used

| | |
|---|---|
| Cell line | L540 |
| Tissue origin | Hodgkin Lymphoma |
| Source | CrownBioscience |
| Growth media | RPMI 1640, 20% FBS |
| Growth conditions | 37° C., 5%, CO2 |

RPMI = Roswell Park Memorial Institute-1640 medium;
FBS = fetal bovine serum

### Implantation of Xenografts

Xenografts were established by subcutaneous (SC) injection of 5×10^{e6} cells suspended in 0.1 mL of PBS into the right front flank of 6-to 8-week-old animals. Tumors were allowed to reach 100-200 mm³ before randomization. Tumors were measured by calliper and the volumes of tumors were calculated using the following formula: Volume (mm3)=(Tumor Length)×(Tumor width)×(Tumor width)/2 Tumor length (the longest tumor dimension); Tumor width (the longest tumor dimension perpendicular to length).

### Randomization

Animals were randomized into groups based on the size of the tumor. Randomization was performed based on “Matched distribution” method (StudyDirector™ software, version 3.1.399.19). The date of randomization is denoted as Day 0. There were no animal substitutions.

### Group Designation and Dose Levels

Control animals were dosed with vehicle (5% v/v DMSO/20% v/v Kolliphor HS15/75% v/v purified water (pH 3.0-3.2)) PO, and vehicle and Compound C treated animals were dosed according to Table 6. Dosing was initiated 1 day upon selection and randomization. On day 21 of dosing, all animals from each received the last dose in the morning. Mice were necropsied 6 h later and flash frozen tumors were collected for PD analysis.

TABLE 6
Outline of treatment groups

| | | Dose level | | Days of | Number of |
|---|---|---|---|---|---|
| Group | Treatment | (1, 2, 3) | ROA | dosing | animals (F) |
| 1 | Vehicle | n/a | PO | 21 | 10 |
| 2 | Compound C | 3 | PO | 21 | 10 |
| 3 | Compound C | 2 | PO | 21 | 10 |
| 4 | Compound C | 1 | PO | 21 | 10 |

F = female;
ROA = route of administration;
PO = oral administration

### Tumor Measurements and Body Weights

Tumors were measured twice weekly by caliper, and the volumes of tumors were calculated using the formula: Volume (mm³)=(Tumor Length)×(Tumor width)×(Tumor width)/2
Relative tumor volume (RTV) was calculated using the formula: RTV for day X=(Tumour volume on day X)/(Tumour volume on day 0)
The anti-cancer effect of Compound C was expressed as percent of tumor growth inhibition (TGI) calculated on last day of the study using the formula:
Percentage TGI on day X for treatment group=(((Geomean RTV Vehicle day X)−(Geomean RTV Treatment group day X))/((Geomean RTV Vehicle on day X)−1)×100
Animals were weighted daily for the dosing phase and twice weekly for all other phases. Percentage of body weight change was calculated using the formula: Percentage body weight change on day X=(((Body weight day X)−(Body weight day of select)×100))

### Assays

### Vehicle Formulation

Kolliphor HS15 was melted in hot water (˜40° C.) and vortex mixed to ensure that the solution was homogenous. Subsequently, DMSO (5% of final vehicle volume) was added in a glass vial. Next, Kolliphor HS15 (20% of final vehicle volume) was added to the glass vial and vortexed mixed well. Up to 80% of final vehicle volume was made with purified water and vortexed mixed well and pH was adjusted to 3.0-3.2 using hydrochloric acid (1 M) and vortexed mixed well. Up to 100% of final vehicle volume was made with purified water and vortexed mixed well.

### Compound C Formulation

Kolliphor HS15 was melted in hot water (˜40° C.) and vortex mixed to ensure solution is homogenous. The appropriate amount of the compound was weighed into glass vial. Subsequently, DMSO (5% of final vehicle volume) was added in a glass vial and vortexed mixed well to fully dissolve the compound. Next, Kolliphor HS15 (20% of final vehicle volume) was added to the glass vial and vortexed mixed well. Up to 80% of final vehicle volume was made with purified water and vortexed mixed well and pH was adjusted 3.0-3.2 using hydrochloric acid (1 M) and vortexed mixed well. Up to 100% of final vehicle volume was made with purified water and vortexed mixed well.

### Pharmacodynamic Analysis

### Western Blot

To determine levels of protein of interest in tumor samples, snap frozen tumor fragments at the end of the PD study were used and protein extracted by adding 600-1000 μl lysis buffer for small and large tumors, respectively. Lysis buffer includes; RIPA buffer (Thermo, #89901), complete protease inhibitor tablets (Roche, #58880600, 2 tablets/50 ml), phosphatase inhibitor cocktail 2 & 3 (Sigma, #P5726, #P0044), with benzonase nuclease (Sigma, #E1014). Samples were homogenised for 30 seconds three times at 6.5 m/s in fast prep machine. Lysates were then sonicated in a chilled diagenode bioruptor for 1 cycle of 30 sec on high and then chilled on ice for 30 minutes. Lysates were centrifuged twice for 10 minutes at 13000 rpm at 4 degrees, with a change of tubes between runs to discard debris. Lysates were transferred to a deepwell plate, and protein in the supernatant measured using BCA normalisation method (Thermofisher, #23225). Protein concentrations were normalised to 45 μg by diluting with 4× Sample Buffer (Invitrogen, #NP007), 10× Reducing agent (Invitrogen, #NP0009) and H₂O. Samples were then boiled for 5 minutes at 95 degrees. Following protein separation on a 4-12% bis tris gel, protein was transferred onto nitrocellulose membranes (Thermofisher #IB21001) using an Iblot2. Primary antibodies that recognised SDMA or vinculin were diluted in 0.05% Tween (TBST)+5% Marvel, and incubated overnight at 4 degrees Celsius. The membranes were washed three times for 15 minutes each in 20 mL of TBST. A secondary rabbit (CST #7074) or mouse (CST #7076) horseradish peroxidase (HRP)-linked antibody was diluted 1:2000 in TBST+5% Marvel and incubated for 1 hour at room temperature. The membranes were washed three times for 15 minutes each in 20 ml of TBST, and signal was detected using chemiluminescent SuperSignal West Dura extended duration substrate (Thermofisher, #34075) and quantified using Syngene software. The 30 kDa molecular weight band of PRMT5's substrate, SDMA, was quantified using Syngene software. The 110 kDa molecular weight band of vinculin was also quantified. Statistical analysis was performed on values normalized to vinculin using ordinary one-way ANOVA compared with vehicle control. SDMA (SDMA #13222 1:1000 dilution was obtained from CST); Vinculin (#V9131 1:10,000 dilution) was obtained from Sigma.

### Statistical Methods

### In Vivo

Tumor volumes were plotted as geometric means with SEM. Percentage of body weight change was plotted as means with SEM. Significant p-values for TGI relative to vehicle treated controls (relative tumor volumes) at the last day of treatment, were obtained from a Mann-Whitney one-tailed test, and calculated by GraphPad Prism 8.4.3

### Pharmacodynamic Analysis (PD)

Primary analysis was carried out using Excel, where raw data was firstly normalised to vinculin, secondly to the geomean of vehicle control and then multiplied by 100. GraphPad Prism 8.4.3 was used for statistical analysis, where data was log transformed (Y=Log (Y)) and an ordinary one-way ANOVA test, adjusting for multiple comparisons (Dunnett), was performed. The mean difference was taken from Prism and used to calculate the percentage inhibition, following this equation:
Untransforming the Mean difference value to percentage=1−((−1*Mean Difference)*10)*100. Significant p-values, if any, obtained from the ANOVA test, were quantified using GraphPad Prism 8.4.3.

SDMA protein levels were measured.

### Results

Compound C exhibited a dose-dependent efficacy at inhibiting tumour growth in vivo (FIG. 14 and Table 7). Dosing at Dose Level 3, Dose Level 2 or Dose Level 1 resulted in tumour growth inhibition (93%, 52%, and 22%, respectively).

TABLE 7
Anti-cancer effect of Compound C

| | | | Day TGI | |
|---|---|---|---|---|
| | | | measured | |
| | | | (end of | p-value t-test vs |
| Treatment | Dose | TGI (%) | treatment) | vehicle control^{a} |
| Compound C | Dose Level 3 | 93 | 21 | *** (p < 0.0001) |
| | Dose Level 2 | 52 | 21 | ** (p = 0.0016) |
| | Dose Level 1 | 22 | 21 | NS (not significant) |

^{a}One tailed Mann-Whitney test calculated for RTV at the last day of treatment

Compound C was well tolerated at all doses tested, there was no significant body weight loss during the treatment period when compared to the vehicle-treated group (FIG. 15).

Pharmacodynamic changes were assessed by western blot for a decrease in SDMA (marker of target engagement) following treatment with Compound C. Dosing of Compound at Dose Level 3, Dose Level 2 or Dose Level 1 resulted in a reduction of SDMA protein levels (99.1%, 97.6%, and 83.9%, respectively; FIG. 16 & Table 8).

TABLE 8
Percentage Inhibition of SDMA treated with Compound C

| | | Percentage Inhibition | |
|---|---|---|---|
| Treatment | Dose | of SDMA | p-value^{a} |
| Compound C | Dose Level 3 | 99.1 | **** (p < 0.0001) |
| | Dose Level 2 | 97.6 | **** (p < 0.0001) |
| | Dose Level 1 | 83.9 | **** (p < 0.0001) |

^{a}ordinary one way ANOVA, adjusted for multiple comparisons (dunnett)

### CONCLUSIONS

Compound C demonstrated dose-dependent efficacy and target engagement in an MTAP silenced subcutaneous Hodgkin lymphoma xenograft model in vivo without causing significant body weight loss compared to the vehicle group.

All references cited herein, including patents, patent applications, papers, text books, and the like, and the references cited therein, to the extent that they are not already, are hereby incorporated herein by reference in their entirety for all purposes.

1. A method of treatment of cancer comprising administering a MTA synergistic PRMT5 inhibitor to a patient in need thereof, wherein the patient has been identified as having a cancer in which wild type MTAP gene has been silenced.

2. A MTA synergistic PRMT5 inhibitor for use in the treatment of cancer, wherein the cancer is characterised as being wild type MTAP gene silenced.

3. A MTA synergistic PRMT5 inhibitor for use in the manufacture of a medicament, wherein the medicament is for use in the treatment of a cancer that is wild type MTAP gene silenced.

4. A pharmaceutical composition comprising a MTA synergistic PRMT5 inhibitor for use in the treatment of cancer, wherein the cancer is characterised as being wild type MTAP gene silenced.

5. A kit comprising a MTA synergistic inhibitor and instructions for its use in the treatment of a MTAP gene silenced cancer.

6. A method of treatment of cancer comprising the steps of i) analysing a sample obtained from a patient in need of treatment that they have a cancer that is wild type MTAP gene silenced; and ii) administering a therapeutically effective amount of a MTA synergistic PRMT5 inhibitor to the patient in need of treatment.

7. A method of treatment, inhibitor for use composition for use or kit for use according to any one of claims 1 to 6, wherein the wild type MTAP gene silenced cancer is selected from bladder cancer, breast cancer, Diffuse Large B-cell Lymphoma (DLBCL), Hodgkin Lymphoma, kidney cancer, leukaemia, lung cancer, Non-Hodgkin Lymphoma, ovarian cancer, pancreatic, sarcoma and skin cancer.

8. A method of treatment, inhibitor for use composition for use or kit for use according to claim 7, wherein the wild type MTAP gene silenced cancer is Hodgkin Lymphoma.

9. A method of treatment, inhibitor for use composition for use or kit for use according to claim 8, wherein the Hodgkin Lymphoma is a classical Hodgkin Lymphoma, such as nodular sclerosing (NSHL), mixed cellularity (MCHL), lymphocyte-rich (LRHL) and lymphocyte depleted (LDHL) or nodular lymphocyte-predominant Hodgkin Lymphoma.

10. A method of treatment, inhibitor for use composition for use or kit for use according to any one of claims 1 to 9, wherein the MTA synergistic PRMT5 inhibitor is a compound of Formula (IV), or a pharmaceutically acceptable salt thereof: 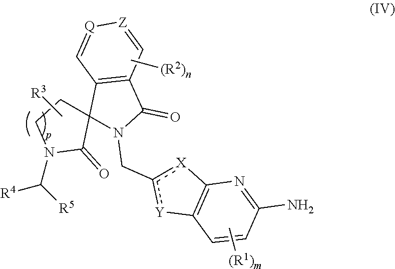
wherein:
the ring containing X and Y is a pyrrole and X is NH and Y is CH or X is CH and Y is NH;
Z is selected from CH, CF, CCl or, if Q is not N, N;
Q is selected from CH, CF, CCl or, if Z is not N, N;
m is 0, 1 or 2;
n is 0, 1 or 2;
p is 1 or 2;
R¹ is in each occurrence independently selected from F, Cl, CN, Me, CF₃, C₁-C₃ alkyl, cyclopropyl, C₁-C₃ fluoroalkyl, OMe or C₁-C₃ alkoxy;
R² is in each occurrence independently selected from F, Cl, Me, MeO and CF₃;
R³ is H, Me, C₁-C₃ alkyl or C₁-C₃ fluoroalkyl;
R⁴ is H, Me or C₁-C₃ alkyl;
R⁵ is H, Me, C₁-C₃ alkyl, C₁-C₃ fluoroalkyl, CH₂OMe, CH₂OCHF₂, CH₂OCF₃, CH₂O(C₁-C₃ alkyl), CH₂O(C₁-C₃ fluoroalkyl), C(CH₂CH₂) R⁶, CCR₇, CH₂R⁸, R³ or CH₂R¹⁰;
R⁶ is H, Me, CH₂F, CHF₂, CF₃, CH₂OH or CH₂OMe;
R⁷ is H, Me, cyclopropyl, C₁-C₃ alkyl, C₁-C₃ fluoroalkyl, C₃-C₆ cycloalkyl or a 5-membered heteroaryl group optionally substituted with Me, C₁-C₃ alkyl, F or Cl;
R⁸ is a 5-membered heteroaryl optionally substituted with Me, C₁-C₃ alkyl, F or Cl;
R⁹ is an optionally substituted phenyl, 5-or 6-membered heteroaryl, or bicyclic heteroaryl group; and
R¹⁰ is an optionally substituted phenyl, 5-or 6-membered heteroaryl, or bicyclic heteroaryl group.

11. A method of treatment, inhibitor for use, composition for use or kit for use according to claim 10, wherein the MTA synergistic PRMT5 inhibitor is(S)-2-((5-Amino-6-fluoro-1H-pyrrolo[3,2-b]pyridin-2-yl)methyl)-5-fluoro-1′-(4-fluorobenzyl)spiro[isoindoline-1,3′-pyrrolidine]-2′,3-dione: 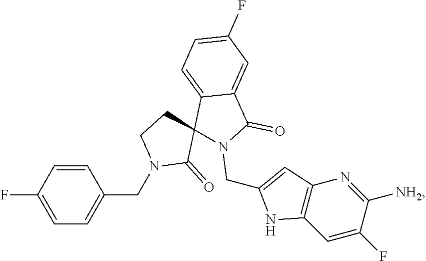 [SMILES: Nc1nc2cc(CN3C(=O)c4cc(F)ccc4[C@]34CCN(Cc3ccc(F)cc3)C4=O)[nH]c2cc1F]
or a pharmaceutically acceptable salt thereof.

12. A method of treatment, inhibitor for use, composition for use or kit for use according to claim 11, wherein the MTA synergistic PRMT5 inhibitor is(S)-2-((5-Amino-6-fluoro-1H-pyrrolo[3,2-b]pyridin-2-yl)methyl)-5-fluoro-1′-(4-fluorobenzyl)spiro[isoindoline-1,3′-pyrrolidine]-2′,3-dione:

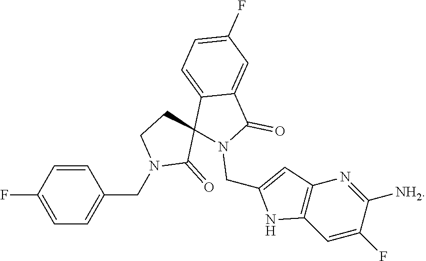 [SMILES: Nc1nc2cc(CN3C(=O)c4cc(F)ccc4[C@]34CCN(Cc3ccc(F)cc3)C4=O)[nH]c2cc1F]

13. A method of treatment, inhibitor for use, composition for use or kit for use according to claim 11, wherein the MTA synergistic PRMT5 inhibitor is a pharmaceutically acceptable salt of(S)-2-((5-Amino-6-fluoro-1H-pyrrolo[3,2-b]pyridin-2-yl)methyl)-5-fluoro-1′-(4-fluorobenzyl)spiro[isoindoline-1,3′-pyrrolidine]-2′,3-dione:

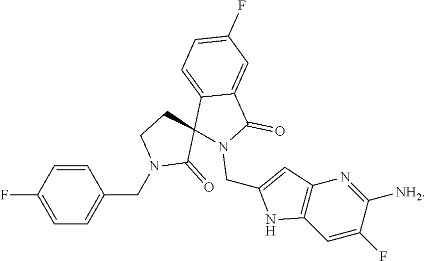 [SMILES: Nc1nc2cc(CN3C(=O)c4cc(F)ccc4[C@]34CCN(Cc3ccc(F)cc3)C4=O)[nH]c2cc1F]

14. A method of treatment, inhibitor for use, composition for use or kit for use according to claim 11, wherein the MTA synergistic PRMT5 inhibitor is(S)-2-((5-Amino-6-fluoro-1H-pyrrolo[3,2-b]pyridin-2-yl)methyl)-1′-(but-2-yn-1-yl)-5-fluorospiro[isoindoline-1,3′-pyrrolidine]-2′,3-dione: 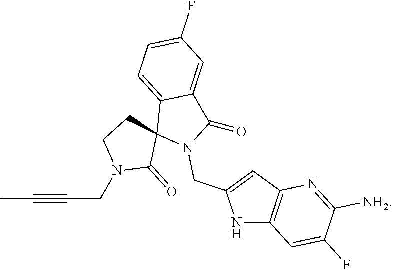 [SMILES: CC#CCN1CC[C@@]2(C1=O)c1ccc(F)cc1C(=O)N2Cc1cc2nc(N)c(F)cc2[nH]1]
or a pharmaceutically acceptable salt thereof.

15. A method of treatment, inhibitor for use, composition for use or kit for use according to claim 11, wherein the MTA synergistic PRMT5 inhibitor is(S)-2-((5-Amino-6-fluoro-1H-pyrrolo[3,2-b]pyridin-2-yl)methyl)-1′-(but-2-yn-1-yl)-5-fluorospiro[isoindoline-1,3′-pyrrolidine]-2′,3-dione:

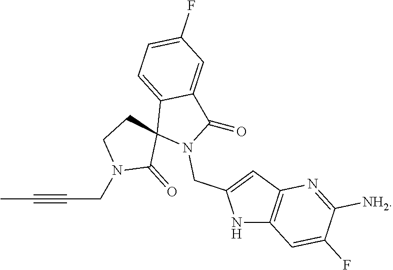 [SMILES: CC#CCN1CC[C@@]2(C1=O)c1ccc(F)cc1C(=O)N2Cc1cc2nc(N)c(F)cc2[nH]1]

16. A method of treatment, inhibitor for use, composition for use or kit for use according to claim 11, wherein the MTA synergistic PRMT5 inhibitor is a pharmaceutically acceptable salt of(S)-2-((5-Amino-6-fluoro-1H-pyrrolo[3,2-b]pyridin-2-yl)methyl)-1′-(but-2-yn-1-yl)-5-fluorospiro[isoindoline-1,3′-pyrrolidine]-2′,3-dione:

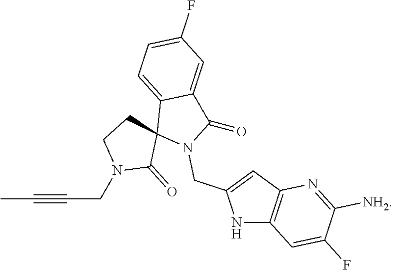 [SMILES: CC#CCN1CC[C@@]2(C1=O)c1ccc(F)cc1C(=O)N2Cc1cc2nc(N)c(F)cc2[nH]1]

17. A method of identifying a patient that will benefit from treatment with a MTA synergistic PRMT5 inhibitor, the method comprising the step of identifying that a sample obtained from the patient is wild type MTAP gene silenced.

18. A method of identifying a wild type MTAP gene silenced tumour comprising the step of identifying that relevant tumour cells in a sample obtained from the patient exhibit reduced MTAP protein in their nuclei and their cytoplasm by performing a immunohistochemical assay for MTAP protein.
